SEQUENCE LISTING (7 sequences)

SEQ ID NO: 1 - DNA, 29 bp, Artificial Sequence
  Description of Artificial Sequence High-affinity probe for inducible element
agcttcatttcccgtaaatccctaaagct

SEQ ID NO: 2 - DNA, 36 bp, Artificial Sequence
  Description of Artificial Sequence Sequence for site-directed mutagenesis of FGFR3 (mouse K644E)
ggactactacaaggagaccacaaacggccggctacc

SEQ ID NO: 3 - protein, 30 aa, Artificial Sequence
  Description of Artificial Sequence Mutated STAT activation sequence of tyrosine kinase FGFR3 (K650E)
DFGLARDVHNLDYYKKTTNGRLPVKWMAPE

SEQ ID NO: 4 - protein, 30 aa, Artificial Sequence
  Description of Artificial Sequence Mutated STAT activation sequence of tyrosine kinase c-kit
DFGLARDIKNDSNYVVKGNARIPVKWMAIE

SEQ ID NO: 5 - protein, 30 aa, Artificial Sequence
  Description of Artificial Sequence Mutated STAT activation sequence of tyrosine kinase RET
DFGLSRDVYEEDSYVKRSQGRIPVKWMAIE

SEQ ID NO: 6 - protein, 30 aa, Artificial Sequence
  Description of Artificial Sequence Mutated STAT activation sequence of tyrosine kinase INRK
DFGMTRDIYETDYYRKGGKGLLPVRWMAPE

SEQ ID NO: 7 - protein, 30 aa, Artificial Sequence
  Description of Artificial Sequence Mutated STAT activation sequence of tyrosine kinase PDGFR-B
DFGLARDIMRDSNYISKGSTFLPLKWMAPE

(19) United States
(12) Patent Application Publication
(10) Pub. No.: US 2003/0105057 A1
(43) Pub. Date: Jun. 5, 2003

(54) Methods and compositions for stimulating apoptosis and cell death or for inhibiting cell growth and cell attachment

(75) Inventors: Xin-Yuan Fu, Guilford, CT (US); Yue E. Chin, New Haven, CT (US); Bing Xie, New Haven, CT (US)

Correspondence Address: MORGAN, LEWIS & BOCKIUS LLP, 1701 MARKET STREET, PHILADELPHIA, PA, 19103-2921, US

(73) Assignee: Yale University

(21) Appl. No.: 10/267,209

(22) Filed: Oct. 9, 2002

Related U.S. Application Data
(63) Continuation of application No. 09/381,308, filed on Feb. 7, 2000, now abandoned.
Application No. 09/381,308 is a 371 of international application No. PCT/US98/05307, filed on Mar. 19, 1998.
(60) Provisional application No. 60/041,410, filed on Mar. 19, 1997.

(51) Int. Cl. A61K 48/00; C12Q 1/68; G01N 33/574; C12N 5/02

(52) U.S. Cl. 514/44; 435/375; 435/6; 435/7.23

(57) ABSTRACT

The present invention relates generally to methods of modulating the rate and/or amount of a cellular process selected from the group consisting of cell growth, cell detachment and cell migration, and cellular apoptosis, said method comprising altering the RECEPTOR/PTK-STAT pathway of a cell. More particularly, the present invention relates to methods wherein the RECEPTOR/PTK-STAT pathway is altered by increasing or decreasing the amount of phosphorylated RECEPTOR/PTK-STAT proteins present in a cell. The present invention also relates to the identification of agents that either promote or inhibit the phosphorylation of RECEPTOR/PTK-STAT proteins, as well as to the agents themselves and to the methods which utilize such identified agents. The methods of the present invention are useful for treating mammalian diseases, including, but not limited to, cancer, autoimmune diseases, viral susceptibility, degenerative disorders, ischemic injuries, and conditions of obesity.

### STATEMENT OF RIGHTS TO INVENTIONS MADE UNDER FEDERALLY SPONSORED RESEARCH

The present invention was developed in part using government funds. The government has certain rights to the present invention. The underlying research was supported by grants from the NIH (RO1 AI 34522).

This application is based on U.S. provisional application No. 60/041,401, which is incorporated in its entirety by reference.

### FIELD OF THE INVENTION

The present invention pertains, in general, to the fields of cell death (apoptosis), cell growth control, and cell attachment. In particular, the present invention pertains to methods and compositions for increasing cell death or apoptosis, and methods and compositions for reducing cell growth or cell adhesion based on the phosphorylation, activation and expression of cellular proteins.

### BACKGROUND OF THE INVENTION

All publications and patent applications herein are incorporated by reference to the same extent as if each individual publication or patent application was specifically and individually indicated to be incorporated by reference.

Apoptosis or programmed cell death is an active process that is essential for normal development and homeostasis in multicellular organisms and provides a defense against viral invasion and oncogenesis (Wyllie et al., 1980; Ellis et al., 1991; Raff, 1992; Steller, 1995; Martin and Green, 1995; White, 1996). It is known that there are a number of independent pathways to apoptosis. For example, p53 is involved in apoptosis in response to DNA damage and other cellular damages (Clarke et al., 1993; Lowe et al., 1993; White, 1996). Certain growth inhibitory cytokines are capable of inducing apoptosis independent of p53. Tumor necrosis factors (TNFs) and Fas can both trigger a cell death (Nagata and Golstein, 1995; Cleveland and Ihle, 1995; Fraser and Evan, 1996). It has been recently shown that TNF/Fas may induce a cascade of proteolytic signaling pathways to mediate apoptosis (Tartaglia et al., 1993; Hsu et al., 1995; Muzio et al., 1996; Boldin et al., 1996; Cleveland and Ihle, 1995; Fraser and Evan, 1996). The key mediators of apoptosis are the ICE (interleukin-1b-converting enzyme) family cysteine proteases (recently renamed as Caspase, see Alnemri et al., 1996), which are related to the C. elegans programmed cell death gene ced-3 (Yuan et al., 1993). The mammalian ICE protease family comprises at least eleven members (Stanger et al., 1995; Fraser and Evan, 1996; Salvesen, 1997). It is possible that these different ICE family members may function in response to the different apoptosis pathways.

In contrast to TNF/Fas, many other growth factors or cytokines can activate receptor protein tyrosine kinase and/or receptor-associated tyrosine kinases (presented here as Receptor/PTK) signaling pathways (Schlessinger and Ullrich, 1992; van der Geer et al., 1994; Ihle and Kerr, 1995). The Receptor/PTK pathways are believed to mediate cell growth and to protect cells from apoptosis (Cleveland and Ihle, 1995; Thompson, 1995). For instance, many kinds of cells can not survive unless the necessary growth factors or cytokines are provided. Thus, growth factors, such as insulin-like growth factor (IGF)-1, EGF and PDGF, which normally induce mitogenic responses, act as survival factors (Bennett et al., 1994; Harrington et al., 1994; Englert et al., 1995; Jung et al., 1996; Thompson, 1995).

It is well-established that growth factors such as EGF can activate a signaling cascade. This cascade links the growth factor receptor tyrosine kinase or receptor associated tyrosine kinases to the Ras protein, then to downstream serine/threonine kinases, such as the members of the MAP kinase family (Schlessinger and Ullrich, 1992; van der Geer et al., 1994). The kinases may translocate to the nucleus and phosphorylate transcription factors such as c-Jun and TCF. However, it is not understood in detail how the cell survival or apoptosis is regulated through this cascade pathway.

Parallel to this kinase cascade signaling pathway, a direct signaling pathway has also been revealed in the past few years. In this pathway, signal transduction is mediated by the protein tyrosine kinases, and their specific substrates: SH2 (Src homology region) containing STAT proteins (Fu, 1992; Schindler et al., 1992; Velazquez et al., 1992; Larner et al., 1993; Muller et al., 1993; Darnell et al., 1994; Fu, 1995a; Ihle and Kerr, 1995). Although this signaling pathway was first revealed in the interferon system, it has been further demonstrated that most cytokines and growth factors, including EGF, PDGF, CSF-1, insulin, IL-2, IL-3, IL-4, IL-5, IL-6, IL-7, and IL-10 etc., can activate the direct STAT signaling pathway (Fu and Zhang, 1993; Larner et al., 1993; Ruff-Jamison et al., 1995; Sadowski et al., 1993; Silvennoinen et al., 1993) (reviewed in Darnell et al., 1994; Fu, 1995; Ihle, 1996). So far more than six members of the STAT protein family have been identified in higher eukaryotes, and these STAT proteins are believed to respond specifically to different cytokine signals (Ihle, 1996). Antibodies and other entities that are specific to the functional domain of a STAT protein and that could possibly be used to selectively modulate the activity of a STAT protein have been identified (Danell et. al., WO 96/20954 (published Jul. 11, 1996)).

The Jak family of tyrosine kinases were initially recognized as activators of STAT proteins (Ihle, 1995; Muller et al., 1993; Velazquez et al., 1992). However, a variety of tyrosine kinases, such as EGF receptor tyrosine kinase and Src kinase have been shown to activate STAT proteins directly and independently of JAK kinases (Fu and Zhang, 1993; Quelle et al., 1995; Yu et al., 1995). It has been further shown that focal adhesion kinase (FAK), FGF receptor tyrosine kinase, and many other tyrosine kinases can also directly activate STAT proteins (Su et al., 1997; inventors' unpublished). Therefore, this is a common pathway from cell surface receptors with intrinsic, and/or associated with, protein tyrosine kinases (these two kinds are presented here as Receptor/PTKs) to STAT proteins (Receptor/PTK-STAT pathway). However, the physiological and cellular functions of the Receptor/PTK-STAT pathway were not well understood. It has been proposed that the JAK-STAT pathway is involved in cell proliferation and transformation (reviewed in Leonard and O'Shea, 1998).

Mammalian cell proliferation is controlled by cytokines and other polypeptide ligands which may produce positive or negative growth signals. For example, epidermal growth factor (EGF) can stimulate proliferation of many cell types (Carpenter and Cohen, 1979; Cross and Dexter, 1991). In contrast, interferons (IFNs) often inhibit cell proliferation (De Maeyer and De Maeyer-Guignard, 1988). Thus many cytokines or growth factors have traditionally been classified into one of these categories of growth stimulator or inhibitor. However, many cytokines have been shown to stimulate growth in one cell type, while inhibiting growth or inducing differentiation in the other cell types (Sporn and Roberts, 1988). A431 cells, a classical system for the study of EGF receptor function for the past decade, were growth-inhibited by EGF (Gill and Lazar, 1981; Bravo et al., 1985). Interleukin-4 (IL-4) is a well-known growth factor for B-cells, but it can evoke strong growth suppression in many tumor cells (Tepper et al., 1989; Toi et al, 1992; and Lahm et al., 1994). The molecular mechanisms for such cell specific responses to cytokines are not well-defined.

In the recent years, the machinery of cell proliferation and the molecular mechanisms of cell cycle control have been analyzed in detail. The cell cycle is controlled by a family of cyclin-dependent kinases (CDKs) which can be negatively regulated by families of CDK inhibitors (Hunter and Pines, 1994; Sherr and Roberts, 1995). One of the well-studied CDK inhibitors is p21 (WAF1/Cip1/CAP1) which, upon binding to CDKs, blocks their activity and causes cell cycle arrest (El-Deiry et al, 1993; Gu et al., 1993; Harper et al., 1993; Xiong et al., 1993; Noda et al., 1994). p21 is induced by the transcriptional activating function of the tumor suppresser protein p53, suggesting its inhibitory role in p53-mediated G1 check-point control (El-Deiry et al., 1993; 1994). However, p21 is also induced by the proliferative signal in T lymphocytes and other growing cells (Firpo et al., 1994; Li et al., 1994; Sheikh et al., 1994). Detailed biochemical analysis has shown that p21 may exist in both active and inactive CDK/cyclin quaternary complexes. The increase of the ratio of p21 to CDK/cyclin may convert the active complex into inactive complexes (Zhang et al., 1994; Harper et al., 1995; reviewed in Hunter and Pines, 1994; Sherr and Roberts, 1995). It is of interest to determine whether any cytokines may play a role in regulation of p21 expression, which may shift the p21:CDK/cyclin ratio, resulting in proliferative or anti-proliferative effects.

It is believed that some of the genes that control the cell cycle are regulated by cytokine-induced signals. However, pathways from cytokine-induced signal transduction to control of cell growth are largely undefined. For example, it is not understood how signals from cytokine receptors are transduced to specific transcription factors regulating expression of genes encoding cell cycle regulators such as p21. It is well-established that growth factors can activate a protein kinase cascade (reviewed in Cantley et al., 1991; Schlessinger and Ullrich, 1992). This cascade links the growth factor receptor associated tyrosine kinase to the Ras protein, then to downstream serine/threonine kinases, such as the members of MAP kinase family. The kinases may translocate to the nucleus and phosphorylate transcription factors such as c-Jun and TCF (Hill and Treisman, 1995; Karin and Hunter, 1995). However, it is not known how the cell cycle machinery is regulated through this cascade pathway.

The properties and functions of a living cell are tightly regulated by extracellular matrix (ECM) proteins and soluble cytokines. A variety of transmembrane receptors, which can specifically interact with these ECM proteins and cytokines, transduce signals into the cell causing cellular effects, such as induction of gene expression. Integrins that are heterodimeric transmembrane receptors, bind the ECM proteins including fibronectin and other cell adhesion molecules (Clark and Brugge, 1995; Hynes, 1992; Schwartz, et al., 1995). Similarly to the signal transduction induced by cytokine:receptor binding, interaction of integrins with the ECM proteins can induce tyrosine phosphorylation of many intracellular proteins. Among them, the focal adhesion kinase (FAK) has been shown to be tyrosine phosphorylated during some integrin-mediated cell adhesion and is believed to play important roles in integrin signal transduction (Guan and Shalloway, 1992; Hanks, et al., 1992; Schaller, et al., 1992). Like receptor tyrosine kinases, FAK interacts with a pool of signaling intracellular proteins, including c-Src, phosphatidylinositol-3 (PI3)-kinase, Grb2 and p130^{CAS} (Schaller, et al., 1994; Schlaepfer, et al., 1994; Chen and Guan, 1994; Cobb, et al., 1994; Polte and Hanks, 1995; Frisch, et al., 1996). Consistent with functions of these signal proteins, recent studies have shown that FAK may be involved in cell survival (Frisch, et al., 1996, Hanks and Polte, 1997).

### SUMMARY OF THE INVENTION

This invention comprises methods of modulating the rate and/or amount of a cellular process selected from the group consisting of cell growth, cell detachment and cell migration, and cellular apoptosis, said method comprising altering the RECEPTOR/PTK-STAT pathway of a cell. More specifically, the present invention provides methods wherein the RECEPTOR/PTK-STAT pathway is altered by increasing or decreasing the amount of phosphorylated RECEPTOR/PTK-STAT proteins present in a cell.

The present invention provides methods wherein the amount of phosphorylated RECEPTOR/PTK-STAT proteins present in the cell is increased or decreased by introducing into the cell a sense or antisense nucleic acid molecule that encodes a tyrosine kinase and/or a RECEPTOR/PTK-STAT protein.

The present invention comprises altering the RECEPTOR/PTK-STAT pathway by, among other possible methods, increasing or decreasing the expression and/or activation of a RECEPTOR in the pathway; increasing or decreasing the amount of STAT in a cell; increasing or decreasing the amount of kinase present in a cell; altering the interaction of STAT with a RECEPTOR in the pathway; altering the interaction of STAT with a PTK; and by altering the interaction among or between the RECEPTORs, the PTKs, and the STATs in the pathway.

The present invention also provides methods of identifying agents which inhibit apoptosis in a cell through the mechanism of blocking the phosphorylation of RECEPTOR/PTK-STAT by a tyrosine kinase comprising the steps of:

a) incubating STAT, or a fragment thereof, and a tyrosine kinase, or a fragment thereof, with an agent to be tested,;

b) determining whether said agent blocks the phosphorylation of STAT, or a fragment thereof by said tyrosine kinase,

wherein the inhibition of STAT phosphorylation indicates the potential to inhibit apoptosis.

In addition, the present invention provides methods of identifying agents which stimulate or promote apoptosis in a cell through the mechanism of stimulating the phosphorylation of RECEPTOR/PTK-STAT by a tyrosine kinase comprising the steps of:

a) incubating STAT, or a fragment thereof, and a tyrosine kinase, or a fragment thereof, with an agent to be tested, and

b) determining whether said agent stimulates the phosphorylation of STAT, or a fragment thereof by said tyrosine kinase,

wherein the promoting of STAT phosphorylation indicates the potential to stimulate or promote apoptosis.

The presents invention also provides methods for determining whether a RECEPTOR/PTK-STAT protein is phosphorylated as well as for correlating apoptosis with the presence and degree of said RECEPTOR/PTK-STAT phosphorylation, wherein an increase of RECEPTOR/PTK-STAT phosphorylation indicates STAT-mediated apoptosis. The presence of elevated levels of RECEPTOR/PTK-STAT proteins is a diagnostic marker of a number of mammalian diseases, including, but not limited to, Thanatophoric Dysplasia Type II, FGF-receptor associated diseases, cancer, metastasis of cancer cells, autoimmune disorders, diabetes, degenerative diseases, aging, and inflammation.

The present invention provides methods for treating mammalian diseases or developmental defects caused by abnormal cell death induction wherein the methods comprise promoting or inhibiting apoptosis by altering the RECEPTOR/PTK-STAT pathway. The present invention further provides methods of treating mammalian diseases or developmental defects caused by abnormal cell death induction wherein the method comprises inhibiting apoptosis by altering the RECEPTOR/PTK-STAT pathway. The present invention provides methods of treating mammalian diseases or developmental defects caused by abnormal cell proliferation wherein the method comprises inhibiting abnormal cell growth by altering the RECEPTOR/PTK-STAT pathway. The present invention provides methods of treating mammalian diseases or developmental defects caused by cell growth retardation wherein the method comprises promoting cell growth by altering the RECEPTOR/PTK-STAT pathway. The present invention also provides methods of treating mammalian diseases or developmental defects caused by abnormal cell detachment wherein the method comprises promoting cell attachment by altering the RECEPTOR/PTK-STAT pathway. The present invention further provides methods of treating mammalian diseases or developmental defects caused by abnormal cell detachment wherein the method comprises inhibiting cell attachment by altering the RECEPTOR/PTK-STAT pathway.

The present invention provides a method for identifying diagnostic agents for measuring RECEPTOR/PTK-STAT activities in order to determine physiological and pathological conditions, wherein the method comprises the steps of:

a) measuring the activity of a RECEPTOR/PTK-STAT protein,

b) determining whether the activity of the RECEPTOR/PTK-STAT protein is associated with a specific phenotype or a specific disease, and

c). examining cellular localization of STAT protein to determine activation of STATs.

The present invention also provides clones that produce exogenous levels of STAT protein in an amount significantly greater than the parental cell lines from which the clones were developed. The invention further provides clones which exhibit significantly faster cell death following serum withdrawal than the cell death of the parental cell line under the same conditions.

The invention also provides a method for identifying agents that block the phosphorylation of RECEPTOR/PTK-STAT comprising the steps of:

a) growing a clone that over-produces STAT proteins in a serum-based growth media,

b) removing the serum from the growing media and concurrently adding the agent of interest,

c) determining whether said agent blocks the phosphorylation of RECEPTOR/PTK-STAT by observing clone cell viability over time.

The invention also provides a method of diagnosing abnormal STAT activation related to mammalian diseases comprising the steps of:

a) isolating and growing test cells from an individual of interest;

b) conducting nuclear staining of the test cells using anti-STAT antibodies;

c) examining the stained nuclei of the test cells to determine whether or not STAT has been translocated into the nuclei of the test cells; and,

d) comparing the extent of STAT translocation into the nuclei of the test cells to that of normal control cells stained in the same manner.

The invention also provides methods of determining the amount of phosphorylated STAT proteins wherein the methods comprise using anti-phospho-tyrosine STAT, such as anti-phospho-tyrosine STAT1.

One skilled in the art can easily make any necessary adjustments in accordance with the necessities of the particular situation.

Further objects and advantages of the present invention will be clear from the description and examples which follow.

### BRIEF DESCRIPTION OF THE FIGURES

FIG. 1. Activation of STAT1 during Integrin-Mediated Cell Adhesion and by FAK.

A. Activation of STAT1 in A431 cells after plated on fibronectin.

B. STAT1 activation was observed in cells transfected with FAK and STAT1.

FIG. 2. Expression of FAK can Cause Cell Apoptosis through Activation of STAT1.

A. Dramatic morphological changes in transfected cells seemed to parallel with STAT1 activation by FAK.

B. A large portion of cells showed bright white spots representing apparent DNA condensation, caused by FAK-STAT1 activation, indicating that induction of apoptosis.

C. A quantitative measurement of apoptotic cells in various transfected cells.

FIG. 3. STAT1 is Essential for Induction of Apoptosis by FAK.

A-B. The wild type (STAT1+/+) fibroblasts, but not STAT1 null (−/−) fibroblasts, undergo apoptosis, or apoptosis induced when STAT1 was re-introduced with FAK.

C-D. U3A-pSG5 cell line which is STAT1 defective, and U3A-STAT1 cells, in which STAT1 has been stably reintroduced were examined for FAK-induced apoptosis, showing that FAK-STAT1 activation is necessary for the induction of apoptosis.

FIG. 4. Both Integrin Signaling and STAT1 are Necessary for Promotion of Apoptosis Under Physiological Conditions Caused by Serum Withdrawal.

A-B. STAT1+/+ or STAT1−/− embryonic fibroblasts were plated on fibronectin (FN) with media containing no serum. STAT1 positive cells were dying faster.

C-D. STAT1 null and wild type cells were dying at the same rate when they were plated on BSA.

FIG. 5. co-expression of each member of HER receptor family, with each member of STAT proteins causes induction of apoptosis.

A. Joint actions of HER1 and each STAT proteins cause cell death.

B. Joint actions of HER2 and each STAT proteins cause cell death.

C. Joint actions of HER3 and each STAT proteins cause cell death.

D. Joint actions of HER4 and each STAT proteins cause cell death.

FIG. 6. STAT activation induced by EGF causes apoptosis.

A. Comparison of STAT activation in HeLa cells vs. A431 cells in response to EGF.

B. Apoptosis induction of these two cell lines correlates with STAT activation.

C. EGF receptor autophosphorylation and activation of the Ras-MAP kinase pathway are normal in both A431 and HeLa cells.

D. Correlation between Receptor/PTK-STAT activation and apoptosis in MDA-MB-468 cells, a breast cancer cell line and A431-R, an A431 variant.

FIG. 7. ICE Expression Correlated with EGF-STAT activation and induced Apoptosis.

A-B-C. EGF induced ICE gene expression in both A431 and MDA-MB-468 cells, but not in HeLa cells (A) which was correlated with STAT activation.

FIG. 8. Jak1 is necessary for induction of apoptosis in response to IFN-γ.

A. DNA binding activity of STAT activation was absent as determined by EMSAs in E2A4 cells.

B. The strong induction of ICE mRNA normally seen upon IFN-γ treatment in the parental HeLa cells was completely abolished in this JAK1 deficient cell line.

C. Bis-benzimide staining showed that E2A4 cells did not apoptose in the presence of IFN-γ.

FIG. 9. Analysis of apoptosis induction in U3A cells, a STAT1-defective cell line and the parental cell line 2fTGH, and STAT1 reintroduced U3A-S 1-2 cells in response to IFN-γ.

A-B. IFN-γ activate STAT, induce ICE mRNA expression, or lead to apoptosis in 2fTGH and the U3A-S1-2, but not U3A cells in response to IFN-γ.

C. The condensed and/or fragmented nuclei were observed in 2ffGH and U3A-S 1-2, but not U3A cells treated with IFN-γ.

FIG. 10. ICE Gene Is necessary for IFN-γ-Induced Apoptosis

A. Normally activated STAT1 in response to IFN-γ in both ICE^{-/-} and ICE^{+/+} cells.

B. Induced DNA fragmentation was significantly reduced in ICE^{+/+} cells compared with that in ICE^{+/+} cells.

FIG. 11. A General Pathway to Induction of Apoptosis through the Joint Actions of a variety of different Receptor/PTKs and STATs.

A. Apoptosis induction through joint actions of TrkA, a nerve trophin receptor, and each of STAT proteins.

B. Apoptosis induction through joint actions of TrkB, a nerve trophin receptor, and each of STAT proteins.

C. Apoptosis induction through joint actions of a EPH protein, a nerve trophin receptor involved in neuron differentiation, and each of STAT proteins.

D. Apoptosis induction through joint actions of Tie2, a receptor involved in angiogenesis and early development etc., and each of STAT proteins; STAT5A was especially active in causing apoptosis.

E. Apoptosis induction through joint actions of FGFR2, a receptor involved in development and angiogenesis etc., and each of STAT proteins.

F. Apoptosis induction through joint actions of FGFR3, a receptor involved in development and angiogenesis etc., and each of STAT proteins.

G. Apoptosis induction through joint actions of Src, a cytoplasmic tyrosin kinase involved in bone development and tumor transformation etc., and each of STAT proteins.

H. Apoptosis induction through joint actions of Lck, a cytoplasmic tyrosin kinase involved in lymphocytes development and function etc., and each of STAT proteins.

I. Apoptosis induction through joint actions of Itk, a cytoplasmic tyrosin kinase involved in lymphocytes development and function etc., and each of STAT proteins.

The mock was the vector alone transfected cells.

FIGS. 12-14. The STAT proteins control the apoptosis induction by default after growth factor withdrawal.

FIG. 12. Expression of the STAT1 protein in mouse embryonic fibroblasts promotes apoptosis by default after serum withdrawal while deficiency of STAT1 protein reduces apoptosis after serum withdrawal

FIG. 13. Expression of the STAT1 protein in Ba/F3, a cell line derived from pro-B cells, promotes apoptosis after serum or growth factor (IL-3) withdrawal.

FIG. 14. Expression of the STAT3 protein in Ba/F3, mouse embryonic fibroblasts promotes apoptosis after serum withdrawal.

FIG. 15. A negative and positive signaling model is proposed to explain the molecular basis responsible for the dual functions of cytokines.

FIG. 16. Receptor/PTK-STAT activation is a broad molecular signal mediating induction of apoptosis, and represent a mechanism of apoptosis induction by default.

FIG. 17. STAT Activation Induced by EGF and IFN-γ is Correlated with Cell Growth Arrest.

FIG. 18. The p21/WAF1 Expression by STATs in Response to EGF and IFN-γ

A. p21-SIEs are regulatory sites of STAT proteins in the p21 gene.

B. p21 Gene Expression is Correlated with STAT Activation in Response to EGF.

FIG. 19. STAT1 is Essential for Induced Cell Growth Arrest. U3A/Control cells which were deficient in STAT1 were not inhibited by IFN-γ but U3A/STAT1α cells were inhibited by IFN-γ.

FIG. 20. STAT1 Activation induced by expression of a mutant TDII FGFR3 receptor.

A. Kinase activities were assessed by an in vitro autophosphorylation for wild type and the TDII mutant FGFR3.

B. Wild type and the TDII mutant FGFR3 were at similar levels.

C. STAT activation assayed using EMSA. Mutant TDII, but not wild type FGFR3 could induce a STAT1 complex.

FIG. 21. STAT1 nuclear translocation, p21/WAF1 induction and cell growth arrest in TDII transfected cells and in chondrocytes from TDII patients.

a. The FGFR3 protein was expressed on the cell surface (brown color).

b. Expression of TDII receptor on the membrane, and localization of STAT1 in the nucleus.

c. The nuclei in the TDII receptor-transfected cells were counter-stained (dark brown, indicated by arrows.

FIG. 22. STAT1 activation by the expression of TDII receptor would induce expression of p21.

a. the p21 mRNA level was particularly enhanced in TDII-transfected cells compared with other transfected cells.

b. p21 protein was enriched in the nuclei in TDII transfected cells as demonstrated by an immunocytochemical stain with anti-p21 antibody (indicated by arrows).

c. The nuclei in the TDII receptor-transfected cells were counter-stained (dark brown, indicated by arrows.

FIG. 23. STAT1 translocation in chondrocytes from TDII affected, but not other control individuals.

a. STAT1 was expressed at a low level in the chondrocytes from a normal control individual, and STAT1 protein was found in the cytoplasm (brown staining of STAT1 was indicated by arrows; the nuclei were counter stained in blue)

b. STAT1 was translocated into the nuclei, and exclusively stained in the nuclei in many chondrocytes from three TDII-affected individuals

c-d. The nuclear staining by the anti-STAT1 antibody in chondrocytes of the TDII affected patient (c) was completely abolished by the specific competitor (d).

FIG. 24. p21 expression in the same TDII-affected chondrocytes.

a. p21 protein was undetectable with an anti-p21 antibody (no brown stain) in normal chondrocytes.

b. p21 expression was clearly observed in the TDII chondrocytes as indicated by brown or darker nuclear stain by the anti-p21 antibodies (indicated by arrows). There were vacuole-like structures in these cells indicating the cell degeneration or apoptosis.

FIG. 25. Activation loop K650E for TDII of FGFR3 and other tyrosine kinase mutations that may be involved in STAT activation.

FIG. 26. STAT1 can interact with FAK in the transfected cells.

The FAK protein was co-immunoprecipitated with the anti-STAT1 antibody. The identity of the HA-tagged FAK was confirmed further by blotting with an anti-FAK antibody. The expression levels of STAT1 protein were also assayed (lower panel).

FIG. 27. STAT:FAK interactions in untransfected cells.

A. The co-immunoprecipitated STAT1 from 293T cells was protein phosphorylated after they had interacted with FAK.

B. A similar observation was also made in A431 cells.

FIG. 28. The specificity of activation of STAT1 by FAK was confirmed by using various STAT1 and FAK mutants. Mutations of the SH2 domain (STAT1-SH2RQ) and of the tyrosine 701 (STAT1-CYF) in STAT1 prevented its activation when co-transfected with FAK.

FIG. 29. STAT1 and FAK co-expression causes cell detachment.

Co-expression of FAK and STAT1 in 293T cells greatly inhibited the cell adhesion on fibronectin. Expression of either STAT1 or mock expression of β-galactosidase, or STAT1-SH2RQ mutant, had less effect.

FIG. 30. STAT1 is required for cell detachment.

A. Re-introduction of STAT1 protein to U3A cells significantly reduced cell attachment to fibronectin.

B. Embryonic fibroblasts derived from STAT1 deficient or from wild type mice were compared. STAT1 null (−/−) cells attach better than STAT1 +/+ cells at different concentrations of plated fibronectin.

C. A picture showing STAT1 wild-type fibroblast cells were detached and aggregated on the plating to FN, whereas STAT1 −/− cells could attach well at the same conditions.

FIG. 31. STAT1 promotes cell migration. STAT1 −/− and STAT1+/+ fibroblasts were further analyzed using Boyden chamber assay for their migration ability. It was found STAT1 positive cells migrate significantly faster than STAT1 negative cells.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The terms “RECEPTOR/PTK-STAT” and “tyrosine kinase-STAT”, and other variants which may be used interchangeably, and have been used throughout this application and claims to refer to expression and/or activation of STAT proteins (including STAT1, STAT3, STAT4, STAT5A/B, STAT6) by kinases, including receptor tyrosin kinases, such as EPH, HER and FGFR families, and cytoplasmic tyrosine kinases, such as FAK, Itk, TIE, and Src families, in response to stimulations caused by a number of polypeptides and their receptors, such as fibronectin/integrin, EGF and FGF families and their receptors. In the other words, the term RECEPTOR/PTK-STAT is used for description of the collective actions and the signaling pathways from these ligands/receptors to protein tyrosine kinases and to STAT proteins and their target genes.

The current inventions in this application are in the fields of cellular functions of RECEPTOR/PTK-STAT signaling pathways in cell death or survival, cell growth retardation or over-proliferation, cell adhesion or detachment, and cell migration.

The methods of the present invention for increasing or decreasing the amount of phosphorylated RECEPTOR/PTK-STAT proteins present in a cell can be performed by: 1) increasing or decreasing the amount of STATs or kinases or receptors expressed and/or activated in the cell; 2) by altering the interaction of STAT with a receptor or a PTK; or, 3) increasing or decreasing the amount of kinase present in a cell. The methods of the present invention are particularly useful in diagnosis and treatments of cancer, metastasis of cancer cells, autoimmune disorders, Thanatophoric Dysplasia Type II (TDII) and other FGFR-associated growth retardation disorders, diabetes, degenerative diseases, aging and inflammation and those listed in items 4)-6) below.

The present invention further provides methods for identifying agents for use in modulating STAT mediated biological and pathological processes. A skilled artisan can readily use the information disclosed herein, particularly the Examples, to develop assays to identify agents for use in modulating STAT mediated activity. Such agents can increase STAT activity or can be used to decrease STAT activity. For example, an agent that blocks the phosphorylation of STAT by a tyrosine kinase can be identified by: a) incubating STAT, or a fragment thereof, and a tyrosine kinase, or a fragment thereof, with an agent to be tested, and b) determining whether said agent blocks the phosphorylation of STAT by said tyrosine kinase. Such methods can be used to identify agents for use in stimulating or blocking apoptosis, cell adhesion/detachment, cell differentiation, and cell growth and is particularly useful as a diagnostic marker of TDII.

Utilizing the results provided below, a skilled artisan can readily practice and develop the diagnostic, screening and therapeutic methods outlined above and in the claims.

The Examples provide detailed scientific results that can be used by a skilled artisan to 1) develop assay methods for identifying agents that modulate RECEPTOR/PTK-STAT mediated biological and pathological processes; 2) develop diagnostic assays to identify RECEPTOR/PTK-STAT mediated biological and pathological processes; and, 3) act as a target for therapeutic agents for use in modulating RECEPTOR/PTK-STAT mediated biological and pathological processes. Specifically, the Examples provide a basis of therapeutic and diagnostic methods for identifying and treating conditions involving abnormal cell apoptosis/survival, cell growth/retardation, and cell attachment/detachment and migration. Techniques and methods which can be used for the above purposes are described in the literature, such as “Current Protocols in Molecular Biology,” John Wiley & Sons, Inc. 1994 and updated versions; “Current Protocols in Immunology,” John Wiley & Sons, Inc. 1994 and updated versions; “Current Protocols in Neural Sciences,” John Wiley & Sons, Inc. 1994 and updated versions, etc.

In detail, as listed in items 1)-10) below, the present invention provides methods for modulating the rate and amount of a cell growth, cell adhesion/detachment and cell migration, and cellular apoptosis. The methods of the present invention are based on the unexpected observation that RECEPTOR/PTK-STAT signaling pathways, in contrast to many other signaling pathways, can act in a negative fashion (see FIG. 15 and FIG. 16 for summary). Specifically, the Examples show that activation of the STAT proteins, including STAT1, STAT3, STAT4, STAT5A/B, STAT6, mediated by the phosphorylation by a cellular kinase, receptor tyrosine kinases and/or cytoplasmic tyrosine kinases and/or other kinases, such as but not limited to, EGFR (Her family), FGFR family, FAK, JAK, Src, Lck, Itk, TIE2, c-kit, RET, INRK, PDGFR-B and other members of the tyrosine kinase family of proteins, in response to polypeptide ligands or by co-expression of PTKs and STATs, cause cell apoptosis, decreases the rate and extent of cell growth and promotes cell detachment and migration. Accordingly, cell survival, proliferation and cell adhesion can be stimulated by blocking the RECEPTOR/PTK-STAT signaling pathways, and cell survival and cell growth and cell adhesion can be reduced by increasing the phosphorylation and activation of RECEPTOR/PTK-STAT signaling pathways. Similarly, the activities of RECEPTOR/PTK-STAT proteins can be used as indicators or markers for detection, measurement, diagnostic analysis of status, potentials and commitment of apoptosis, cell proliferation, and cell adhesion/detachment and cell migration.

The following items 1)-10) further explain various aspects of the present invention:

1) The first aspect of this invention is based on the unexpected discovery that activation of RECEPTOR/PTK-STAT causes apoptosis. This invention provides general methods, compositions and procedures for development of diagnostic agents for detection and assaying of apoptosis through measuring activities and/or expression of RECEPTOR/PTK-STAT; and for development of therapeutic agents for either inhibiting or stimulating, induction of apoptosis through interfering with the RECEPTOR/PTK-STAT signaling pathways.

In particular, it is the object of this invention to provides a pharmaceutical and gene-therapeutic methods and composition for treating mammalian diseases or developmental defects caused by abnormal cell death induction or reduction through either promoting or inhibiting apoptosis through interfering with the RECEPTOR/PTK-STAT signaling pathways.

Furthermore, this invention provides methods of utilizing RECEPTOR/PTK-STAT proteins, for developing and designing screening protocols for pharmaceutical (a natural or synthetically produced) and gene-therapeutic agents that can affect activities of STATs and Receptor/PTKs to control apoptosis, and provide diagnosis and treatment of apoptosis-related mammalian diseases or developmental defects through interfering with the RECEPTOR/PTK-STAT signaling pathways.

2) A second aspect of this invention is based on the unexpected discovery that activation of RECEPTOR/PTK-STAT causes cell growth arrest and inhibition of cell proliferation. This invention provide general methods, compositions and procedures for development of diagnostic agents for detection and assaying of cell proliferation or growth retardation through measuring activities and/or expression of RECEPTOR/PTK-STAT; and for development of therapeutic agents for either inhibiting or stimulating, induction of cell proliferation or growth retardation through interfering with the RECEPTOR/PTK-STAT signaling pathways.

In particular, it is the object of this invention to provide a pharmaceutical and gene-therapeutic methods and composition for treating mammalian diseases or developmental defects caused by abnormal cell proliferation or growth retardation, with induction or reduction abnormal cell growth through interfering with the RECEPTOR/PTK-STAT signaling pathways.

Furthermore, this invention provides methods of utilizing RECEPTOR/PTK-STAT proteins, for developing and designing screening protocols for pharmaceutical (a natural or synthetically produced) and gene-therapeutic agents that can affect activities of STATs and Receptor/PTKs to control cell proliferation, and provide diagnosis and treatment of cell proliferation-related mammalian diseases or developmental defects through interfering with the RECEPTOR/PTK-STAT signaling pathways.

3) A third aspect of this invention is based on the unexpected discovery that activation of RECEPTOR/PTK-STAT causes cell detachment and cell migration enhancement. This invention provides general methods, compositions and procedures for development of diagnostic agents for detection and assaying of cell detachment or adhesion and cell migration through measuring activities and/or expression of RECEPTOR/PTK-STAT; and for development of therapeutic agents for either inhibiting or stimulating, induction of cell detachment or adhesion and cell migration through interfering with the RECEPTOR/PTK-STAT signaling pathways.

In particular, it is the object of this invention to provide a pharmaceutical and gene-therapeutic methods and composition for treating mammalian diseases or developmental defects caused by abnormal cell detachment or adhesion and cell migration, with induction or reduction of abnormal cell adhesion/detachment and/or cell migration through interfering with the RECEPTOR/PTK-STAT signaling pathways.

Furthermore, this invention provides methods of utilizing RECEPTOR/PTK-STAT proteins, for developing and designing screening protocols for pharmaceutical (a natural or synthetically produced) and gene-therapeutic agents that can affect activities of STATs and Receptor/PTKs to control cell attachment and cell migration, and provide diagnosis and treatment of mammalian diseases or developmental defects caused by abnormal cell adhesion or detachment and/or cell migration through interfering with the RECEPTOR/PTK-STAT signaling pathways.

4) The inventions in this application provide diagnostic and therapeutic methods for studies and treatments of the diseases and abnormalities that may be associated with inhibition or reduction of apoptosis include, but are not limited to, the following (partly reviewed in Thompson, Science, 267, 1456-1462):

i. Cancer, such as breast, prostate, ovarian and colon cancer; leukemia, such as acute leukemia, follicular lymphophomas; carcinoma with p53 mutations.

ii. Autoimmune diseases with overactive, abnormally produced and increased number of lymphocytes due to less apoptosis, such as arthritis, diabetes, multiple sclerosis and asthma etc, due to over-active lymphocytes, lupus erythematosus, glomerulonephritis.

iii. Less resistance and restriction to viral (including herpes viruses, poxviruses, adenoviruses etc.) infections due to less apoptosis.

iv. Conditions of obesity caused by increased number of adipocytes and loss of feedback control; cardiovascular diseases due to less apoptosis such as atherosclerosis.

In the above conditions and diseases, treatments can be provided by induction of apoptosis by introducing and activating RECEPTOR/PTK-STAT proteins. Additionally, it is desirable to provide expression of the proteins with an agent which targets the target cells, such as an antibody specific for a surface protein on the target cell, a ligand for a receptor on the target cell, etc. Furthermore, pharmaceutical (a natural or synthetically produced) and gene-therapeutic agents that can enhance activities of STATs and Receptor/PTKs to induce apoptosis can be searched, screened, developed, and assayed (see below).

5) The inventions in this application provide diagnostic and therapeutic methods and compositions for studies and treatments of the diseases and abnormalities that may be associated with increased apoptosis include but not limited to the following:

i. Degenerative disorders, in particular, the neurological abnormalities, developmental defects, and aging which are due to cell death resulting from overly active RECEPTOR/PTK-STAT pathways, and/or by default after survival signal reduction and/or deprivation (examples and their mechanisms are presented and discussed in FIGS. 12 to 16 above). These may include but not limited to: Alzheimer's disease, Parkinson's disease, cerebellar degeneration, neuronal damage in multiple sclerosis, diabetes mellitus type I, cartilage destruction such as in rheumatoid arthritis, sepsis and septic shock such as adult respiratory distress syndrome)

ii Ischemic injuries, such as stroke, myocardial infarction, and other related cardiovascular disorders due to too much apoptosis.

iii. Viral infection induced cell death, such as AIDS by HIV, causing elimination of special lymphocytes.

iv. Cell apoptosis during inflammatory responses, due to overly-active RECEPTOR/PTK-STAT signaling pathways, such as those cell death caused by cytokines, antigen receptors, and other cell surface receptors. Cell apoptosis due to cachexia associated with chronic disease, and to Mycobacterium tuberculosis, gastritis, and Helicobacter pylori, etc. Cell apoptosis after toxic stress, such as alcohol, generation of reactive oxygen species, radiation, chemotherapeutical compounds, and other apoptosis inducing or activating agents.

In the above conditions and disorders, diagnosis can be provided by assaying the activities of RECEPTOR/PTK-STAT proteins; and treatments can be provided by prohibition of apoptosis by reducing and/or inactivating RECEPTOR/PTK-STAT proteins. Additionally, it is desirable to provide expression of the proteins with an agent which targets the target cells, such as an antibody specific for a surface protein on the target cell, a ligand for a receptor on the target cell, etc. Furthermore, pharmaceutical (a natural or synthetically produced) and gene-therapeutic agents that can inhibit activities of STATs and Receptor/PTKs to prevent apoptosis can be searched, screened, developed, and assayed (see below).

6) The inventions in this application provide diagnostic and therapeutic methods for studies and treatments of the diseases and abnormalities that may be associated with increased cell proliferation, and cell detachment and cell migration such as a variety cancers, tumor cell metastasis, and invasion during the later stages of cancer development. For these abnormalities, diagnosis and treatments can be provided by induction of apoptosis by introducing and activating RECEPTOR/PTK-STAT proteins. Furthermore, pharmaceutical (a natural or synthetically produced) and gene-therapeutic agents that can enhance activities of STATs and Receptor/PTKs to induce apoptosis can be searched, screened, developed, and assayed (see below).

7) The inventions in this application provide methods and compositions for enhancing the RECEPTOR/PTK-STAT signaling pathways in their functions during cell apoptosis, growth arrest, and cell detachment which include but not limited to the following:

Generating and expressing functional RECEPTOR/PTK-STAT proteins are as described in trade books such as Molecular Cloning, A Laboratory Manual (2nd Ed., Sambrook, Fritsch and Maniatis, Cold Spring Harbor), and Current Protocols in Molecular Biology (Wiley-Interscience, NY, N.Y., 1996). Currently available systems include, but are not limited to, expression in bacteria such as E. coli and eukaryotes such as yeast, baculovirus, or mammalian cell-based expression systems such as using CHO cells, etc.; in vivo delivery systems include but not limited to retrovirus or other viral delivery systems, such as modified and specially engineered viral vectors derived from adenovirus, herpes simplex virus, avipox virus etc.; electroporation and lyposome, such as lipofectin (Life-Sciences) mediated fusion, CaPO4 and DEAE-Dextran transfections etc. Various other delivery techniques can be used for providing the subject compositions at the site of interest, such as injection, use of catheters, trocars, projectiles, pluronic gel, stents, sustained drug release polymers or other device which make local and internal access. These expression and delivery systems provide methods to introduce RECEPTOR/PTK-STAT proteins in vitro into tissue culture cells and in vivo into mammals, for therapeutic purposes.

Additionally, constitutively activated RECEPTOR/PTK-STAT proteins, such as TDII FGFR receptor in FIGS. 20-22, and the kinases listed in FIG. 25. and constitutive activated STAT proteins can be introduced into cells and mammals using the methods described above.

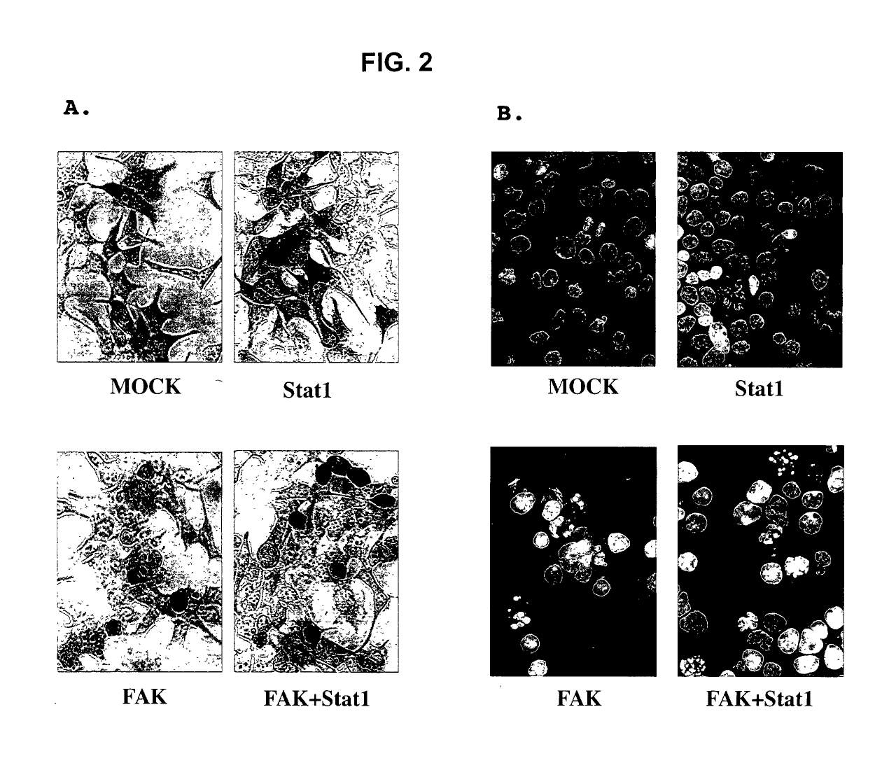

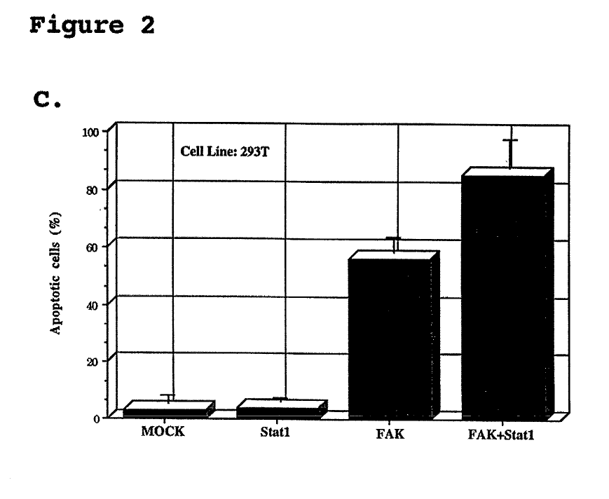

Furthermore, RECEPTOR/PTK-STAT protein-expression vectors may be incorporated into recombinant cells for expression and screening, cell lines and transgenic animals for functional studies (e.g. the efficacy of candidate compounds and other agents and their effects on disease- and/or functional-associated RECEPTOR/PTK-STAT protein activities as regards cell apoptosis/survival, proliferation/retardation, adhesion/detachment and migration). Some of these examples are shown in FIGS. 2-5, FIG. 11, FIGS. 13-14. FIGS. 20-22, FIG. 26, FIGS. 28-30A, etc. These expression systems also provide methods for searching for partner proteins or antagonist proteins or molecules which may enhance RECEPTOR/PTK-STAT functions, which include the yeast two hybrid system, GST-fusion proteins, in vitro translation assays, and co-immunoprecipatation assays (FIGS. 26-27) etc.

8) The inventions in this application provide diagnostic and therapeutic methods and compositions for inhibiting the RECEPTOR/PTK-STAT signaling pathways in their functions during cell apoptosis, growth arrest, and cell detachment which include but not limited to the following:

Expression of antisense molecules of RECEPTOR/PTK-STAT proteins; using dominant negative constructs such as STAT1-CYF used in FIG. 28, kinase dead mutant proteins, and intra-cellular-domain truncated receptors etc. These antagonist molecules can be expressed and delivered into in vitro and in vivo cell and mammal systems using methods described above in item 7. Furthermore, peptides selected from combinatorial peptide libraries and/or represent the interaction domains of the RECEPTOR/PTK-STAT interactions, tyrosine phosphorylated peptides binding to SH2 domains of STATs, and antagonists for STAT DNA binding, such as SIE-like oligonucleotides which have high affinity with STAT DNA binding domain, and STAT-inhibitor proteins or other molecules etc, which can be used as antagonists for inhibition of RECEPTOR/PTK-STAT signaling pathway and its function in induction of apoptosis, growth arrest, and cell detachment.

9) The inventions in this application provide methods and compositions for in vitro and in vivo systems and methods for screening compounds and other agents which can affect, inhibiting or stimulating, the RECEPTOR/PTK-STAT signaling pathways causing either negative or positive effects on cell survival, proliferation, and adhesion/detachment.

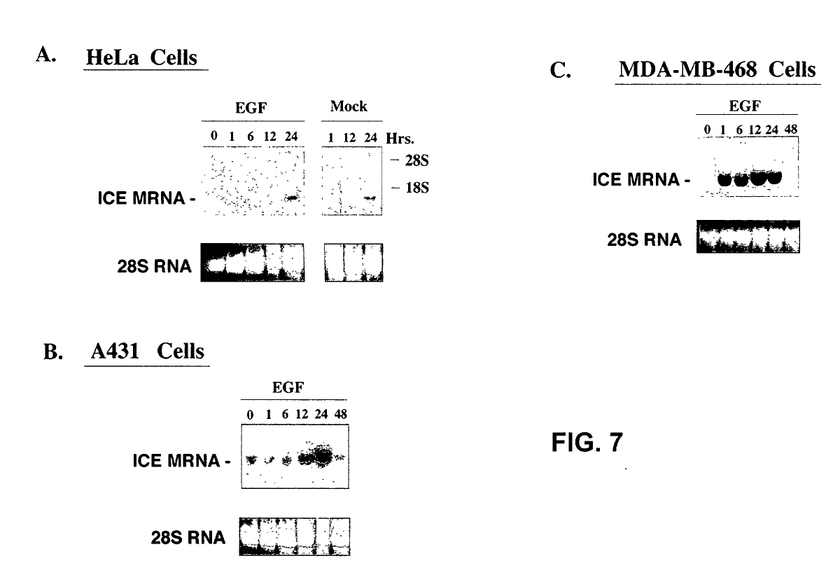

The inventions and cell systems listed in examples and discussed above provides efficient methods of identifying pharmacological agents or lead compounds for agents active in interfering with the RECEPTOR/PTK-STAT signaling and their effects on cells. The SH2 domains of STATs, interactive domains of receptor with STAT or PTKs, STAT-DNA binding site etc. can be targets of these agents. The methods are upgraded to automated, cost-effective high throughput drug screening and should have immediate applications to drug discovery. Target therapeutic indications can be provided by cellular function changes of the RECEPTOR/PTK-STAT signaling and alterations of target genes (such as those observed in FIG. 7 and FIG. 18). The readouts can be as the expression of target genes or DNA binding to their regulatory DNA elements, such as CASPASES or p21 and the SIE identified in their gene promoters (see FIG. 18), and/or using the reporter constructs (such as gene encoding luciferase) linked with their promoters of the targeted genes.

The cellular readout for effective compounds or other agents are rates of cell death induction, cell proliferation and cell attachment. Altered resistant or sensitive cells are isolated by feeding the cells with these agents.

The systems for screening antagonist agents or other negative or positive effectors may use full proteins or key domains of proteins of RECEPTOR/PTK-STAT and their partners in signaling and target gene induction. The candidate agents can be mixtures which include a nucleic acid comprising a sequence which shares sufficient sequence similarity with a gene or gene regulatory region to which it may produce negative or positive effects on RECEPTOR/PTK-STAT functions and their interactions among each other or with other partners. The assay mixture may also comprise a candidate gene therapeutical and pharmacological agent. Typically a plurality of assay mixtures are analyzed in parallel with different agents with a variety of testing concentrations to obtain a differential responses to the various readout systems (see above). Candidate pharmaceutical agents include numerous chemical classes, such as organic compounds; preferably small organic compounds. Small organic compounds have a molecular weight of more than 50 yet less than about 2,500. Agents with chemical groups necessary for structural interactions with proteins and/or DNA include but not limited to an amine, carbonyl, hydroxyl or carboxyl group, and their derivatives, preferably with at least two of the functional chemical groups, more preferably at least three. The candidate agents often comprise cyclical carbon or heterocyclic structures and/or aromatic or polyaromatic structures substituted with one or more of the aforementioned functional groups. Other kinds of agents include but not limited to biomolecules including peptides, saccharides, fatty acids, sterols, isoprenoids, purines, pyrimidines, derivatives, structural analogs or combinations thereof, and so on. Candidate agents can be found and screened from a wide variety of natural or synthetic sources including libraries of synthetic compounds, expression of randomized oligonucleotides, or natural compounds selected from bacterial, fungal, plant and animal extracts. Natural and synthetically selected libraries and compounds are readily modified through conventional chemical, physical, and biochemical means, such as acylation, alkylation, esterification, amidification, etc., to produce more effective structural analogs.

Other components of mixtures include reagents like salts, buffers, neutral proteins, detergents, etc. which may be used to facilitate optimal protein-protein and/or protein-nucleic acid binding and/or reduce non-specific or background interactions, etc. Also, reagents that otherwise improve the efficiency of the assay, such as protease inhibitors, nuclease inhibitors, antimicrobial agents, etc. are also considered and can be selectively used.

10) The inventions in this application provide methods and compositions for the development and discovery of diagnostic agents for measuring RECEPTOR/PTK-STAT activities in order to determine physiological and pathological conditions associated with a phenotype or specific diseases. Examples are shown in FIGS. 20-24. Diseases and abnormalities listed above in item 4), item 5), and item 6) can be diagnosed by measuring the activities of the RECEPTOR/PTK-STAT proteins. The assays, in vivo and in vitro, for detection, and measurement of activities of receptor, PTK and STATs are provided such as EMSA, kinase phosphorylation, in vitro and in vivo protein-protein binding assays, target gene expression, cellular location of STAT, phosphorylation statues as assayed by anti-phosphotyrosine-STAT antibodies (FIG. 27), antibodies against RECEPTORs. PTKs, STATs, and target proteins presented in Figures and examples in this application, and by using epitope tagged, such as Flu-HA tag, Myc tag, Flag-tag (Kodak) and all kinds of commercial available green fluorescent protein tags, etc. and corresponding reagents and assay systems to detect these tagged proteins.

### EXAMPLE 1

In Example 1, in contrast to the conventional view that the RECEPTOR/PTK are promote cell proliferation, the unexpected observations and evidence are presented showing that the STAT pathway initiated by Receptor/PTK activation induces apoptosis. It was demonstrated that expression of a member of a variety of tyrosine kinases, in combination with each of six different STAT proteins, can cause apoptosis both in cultured cells and/or in ligand-stimulated cells. These observations and experiments provide methods and compositions for identifying and developing therapeutic agents for use in modulating Receptor/PTK-STAT mediated physiological and pathological processes.

Materials and Methods

Cell Culture, Extracts, Antibodies and Mobility Gel Shift Assay. For integrin/FAK related experiments, tissue culture plates were coated overnight with 10 ug/ml human plasma fibronectin (Gibco) in PBS, washed twice with PBS and then incubated with 2 mg/ml heat-inactivated (1 hr at 70° C.) BSA in PBS for 2 hrs at 37° C. Cells were harvested by brief trypsinization and washed twice with PBS containing 0.5 mg/ml soybean trypsin inhibitor (Sigma). The cells were resuspended in DMEM without serum and added to coated plates (100 mm) at 8×10⁶. After various times of incubation at 37° C., cells were washed twice with cold PBS and lysed in whole-cell-extract (WCE) buffer (15 mM Hepes, pH 7.9, 400 mM NaCl, 0.5% NP-40, 10% Glycerol, and 1 mM EDTA) containing a cocktail of protease and phosphatase inhibitors (0.5 mM PMSF, 1 mg/ml leupeptin, 1 mg/ml aprotinin, 1 mg/ml pepstatin, 1 mM vanadate, 10 mM NaF, and 1 mM DTT), left on ice for 45 min., centrifuged for 10 min. at 4° C. WCE containing the same amount of total proteins were subjected to EMSA with 10 fmol of ³²P-labeled high-affinity SIE probe. (5′-AGCTTCATTTCCCGTAAATCCCTAAAGCT-3′) (SEQ ID NO. 1) (Chin, et al., 1996)

A431, MDA-MB-468 and HeLa cells (ATCC) were grown in monolayer at 37° C. in Dulbecco's Modified Eagle's Medium (DMEM) supplemented with 10% fetal bovine serum (FBS) or calf serum. 2fTGH and U3A cells, obtained from Dr. G. Stark lab, were grown in DMEM supplemented with 10% FBS and 400 μg/ml hygromycine. U3A-S 1-2 cells were grown in DMEM supplemented with 10% FBS and 400 μg/ml G418. Whole cell extracts were prepared as described previously (Chin et al., 1996). Briefly, cells were starved overnight and treated with 50-200 ng·ml⁻¹ EGF (Gibco) or 10 ng·ml⁻¹ IFN-γ (Genzyme) for 30 min. PBS rinsed cells were lysed in 20 mM Hepes (pH 7.9) buffer containing 0.2% NP-40, 400 mM NaCl, 0.1 mM EDTA, 10% glycerol, 1 mM dithiothreitol (DTT), 1 mM sodium vanadate, 0.5 mM phenylmethylsulphonyl fluoride, 1 μg/ml each of aprotinin, leupeptin and pepstatin. After 30 min gently agitation at 4° C., the supernatants were collected by centrifugation. For all electrophoretic mobility shift assays (EMSAs), M67-SIE was used as the probe (10). DNA-protein binding reactions (15 μl) were performed by incubation of the whole cell extracts in 10 mM Hepes (pH 7.9), 50 mM NaCl, 0.1 mM EDTA, 5% glycerol, 50 μg/ml poly(dI-dC) (Pharmacia), 0.5 mM DTT, and 0.01% NP-40 for 10 min at room temperature, followed by an additional 30 min incubation with ³²P-end-labeled M67 SIE probe (0.1 ng) at room temperature. DNA-protein complexes were separated on 6% non-denaturing acrylamide gels in 0.5XTBE and detected by autoradiography. Anti-EGF receptor antibody was purchased from Gibco and a purified anti-phosphotyrosine polyclonal antibody was a generous gift from Jun-Lin Guan (Cornell University). The EGF receptor immunoprecipitation (IP) and phosphotyrosine antibody blotting were performed as previously described (Fu and Zhang, 1993). Anti-MAP kinase (ERK-2) and anti-ICE (p10) antibodies were from Santa Cruz Biotech, Inc. and the Western blot assays using these two antibodies were performed according to the manufacture's protocol.

Analysis of Apoptontic Cells.

1) Morphological changes in the nuclear chromatin of cells undergoing apoptosis were detected by staining with the DNA-binding fluorochrome bis-benzimide (Hoechst 33258; Sigma). Briefly, monolayer cells (3-6×10⁵) were grown in 6-well plates, and treated with or without EGF (100-200 ng/ml) or IFN-γ (80-160 ng/ml) in the presence of 1% calf serum or fetal bovine serum for different times. After treatment, cells were collected and pelleted at 300×g for 5 min. and washed once with PBS. Cells were resuspended in 100 μl of 3% paraformaldehyde in PBS and incubated for 15 min. at room temperature. After fixation, the cells were washed once with PBS and were stained with 15 μl of bis-benzimide (16 μg/ml) in PBS. Following 15 min. incubation at room temperature, a 5 μl aliquot of cells was placed on a glass slide, and the average number of nuclei per field was scored for the incidence of apoptotic chromatin changes under a fluorescence microscope. Cells with three or more condensed chromatin fragments were considered apoptotic.

2) X-gal analysis and Apoptosis assays: Forty eight hrs after transfection, cells were fixed by 1% glutaraldehyde (in PBS) in 37° C. for 15 min. Cells were stained with 0.2% X-gal (Amersham) (Buffer: 10 mM Na₃PO₄(pH 7.0), 150 mM NaCl, 1 mM MgCl₂, 3.3 mM K₄Fe(CN)₆, 3.3 mM K3Fe(CN)6) for 1 hr. Wash with 70% ethanol, then cover cells with PBS.

3) For TUNEL assay, cover-slip coated with fibronectin (10 ug/ml) in 6-well plate in 4° C. overnight. 1.7×10⁵ cells were seeded for overnight. After 48 hrs of transfection, cells were fixed with 3% paraformaldehyde for 10 min. in room temperature. ApopTag Kit (ONCOR) was used for in situ apoptosis detection according to the company's instructions.

Northern Blot Analysis. Total RNA was prepared with an RNA isolation kit from Gibco-Life Science. RNA (40 ug) was analyzed by electrophoresis in a 1.2% agarose-formaldehyde gel and transferred to a nylon membrane (Zeta-Probe, Bio-Rad). Hybridization was performed at 65° C. overnight in 0.25 M Na₂PO₄ (pH 7.2), 7% SDS, 1 mM EDTA. The wash was performed at 65° C. in 0.04 M Na₂PO₄ (pH 7.2), 1% SDS. The probes (ICE cDNA and CPP32 cDNA) were labeled with a random primed DNA labeling kit (Boehringer-Manheim).

Primary cell preparation, cell viability and DNA fragmentation assay. Mouse (ICE+/+ or ICE−/−) spleens washed with PBS twice and chopped up with a sterilized blade. The chopped spleen cells were then treated with 1×trypsin in EDTA at 37 C. for 10 min. The trypsinized spleen cells were then suspended in RPMI medium supplemented with 10% FBS, 100 U of penicillin per ml, and 100 ng of streptomycin sulfate per ml. After standing in a 15-ml tube for 1-2 min., the suspended single cells were collected and maintained at a concentration of approximately 5×10⁶ cells per ml. Cell viability was determined by trypan blue exclusion after 48 hrs treated with or without IFN-γ. To examine DNA fragmentation, approximately 1×10⁷ cells were seeded in a 100-mm dish and treated with IFN-γ-(50U/ml) or untreated. After 48 hrs treatment, cells were harvested, washed with cold PBS twice and used for DNA isolation. 0.6 ml lysis buffer (10 mM TrisHCl, pH 7.5, 10 mM EDTA, 0.2% Triton-X100) was added to the cells and the lysis was allowed to proceed at room temperature for 15 min. and then centrifuged for 10 min. at 12,000 rpm. The supernatant was collected and mixed with equal volume of phenol and centrifuged for 10 min. at 12,000 rpm. The supernatant was adjusted to 300 mM NaCl and added with 2 volume of ethanol to precipitate DNA. After centrifugation for 10 min. at 12,000 rpm, the DNA pellet was resuspended in 201 TE buffer and digested with 0.2 g RNAse at 37° C. for 30 min. The fragmented DNA was analyzed by running a 2% agarose gel staining with ethedium bromide.

Results

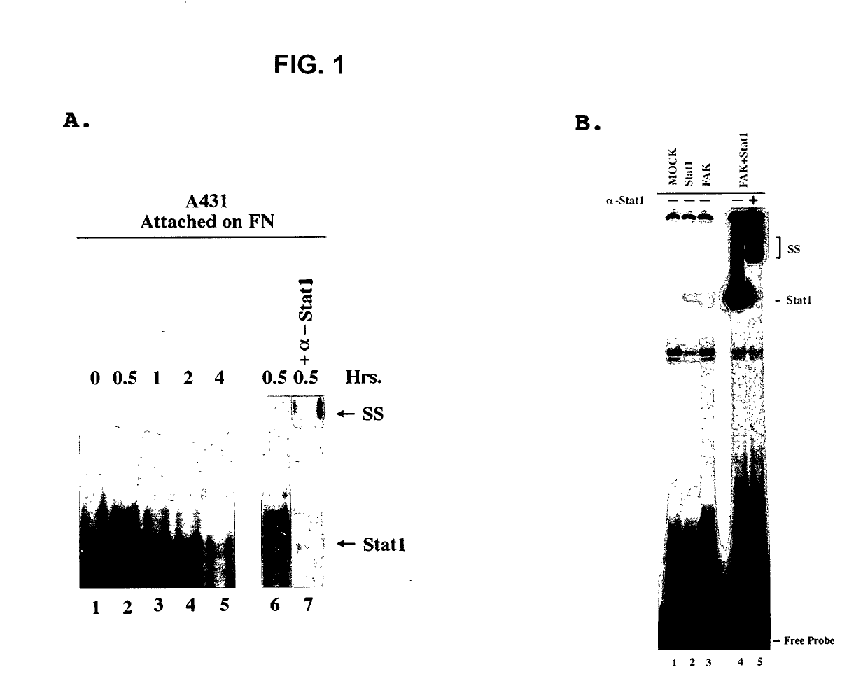

Activation of STAT1 during Integrin-Mediated Cell Adhesion and by FAK. It was found that low levels of STAT1 activity were immediately and transiently enhanced in human A431 cells after plating on fibronectin, a ligand for integrin (FIG. 1A). Activation of STAT1 after plated on fibronectin was clearly observed at the time point of 0.5 hour. The nature of STAT1 in this complex was confirmed when this induced complex was recognized by a STAT1 specific antibody, generating a suppershifted complex (indicated by SS). The STAT1 activity was significantly reduced after 4 hours when cells became attached. These results suggested that STAT proteins may be activated through integrin-activated tyrosine kinase(s) in this condition in vivo.

Since focal adhesion kinase (FAK) is a major tyrosine kinase activated during integrin signaling, 293T cells that were transfected with vectors expressing FAK and STAT1, separately or in combination were further examined (FIG. 1B). STAT1 activation was observed in cells transfected with FAK. but not in mock transfected cells, suggesting that FAK activated endogenous STAT1 in vivo in these cells. Transfection of a HA-tagged STAT1(Fu and Zhang, 1993) also generated a weak STAT1 complex, which migrated slightly slower than endogenous STAT1 complex possibly due to the added HA-tag in the protein. However, in cells co-transfected with FAK and STAT1, STAT1 was strongly activated. This STAT1 complex was recognized by an anti-STAT1 antibody, forming a supershifted complex (SS) in the EMSA.

Expression of FAK can Cause Cell Apoptosis through Activation of STAT1. It was observed that dramatic morphological changes in transfected cells seemed to parallel with STAT1 activation by FAK (FIG. 2A). Since these cells were cotransfected with a vector that expressed b-galactosidase, transfectants could be specifically recognized by the blue color after X-gal staining. Cells that were mock transfected or transfected with STAT1 alone, had little change on cell morphology. However, cells that had been co-transfected with FAK and STAT1, clearly lost cell spreading and were detached from the plate. For the cells transfected with FAK alone, a portion of transfected cells also underwent the similar morphological alterations which might result from the endogenous STAT1 activity induced by FAK.

To confirm that cell morphological alterations caused by FAK-STAT1 activation may induce apoptosis, cells were fixed with paraformaldehyde, then stained with DNA-specific fluorochrome bis-benzimide, and examined by fluorescence microscopy (FIG. 2B). A large portion of cells showed bright white spots representing apparent DNA condensation, a hallmark of apoptosis. These condensed DNA spots coincided with DNA ladders assayed on an agarose gel electrophoresis (data not shown). However, the induction of DNA condensation and fragmentation was not observed in cells transfected with mock or STAT1 alone or mutant FAK with STAT. Consistent with morphological changes, a portion of FAK-alone transfected cells were also apoptotic.

A quantitative measurement of apoptotic cells in various transfected cells (only those cells stained blue due to co-expression of b-galactosidase were counted) based on cell morphology was shown (FIG. 2C). The results were derived from three repeated experiments.

STAT1 is Essential for Induction of Apoptosis by FAK. To confirm the role of STAT1 in FAK-induced apoptosis, the embryonic fibroblasts isolated from STAT1 null or control mice (Durbin, et al., 1996) were subjected to the further analysis. Consistent with the above results with 293T cells, exogenously expressing FAK with STAT1 or FAK alone resulted in dramatic morphological changes, indicating possible apoptosis, which was further confirmed by the TUNEL assay, in the wild type (STAT1+/+) fibroblasts. In contrast, expression of STAT1 alone or in mock transfected cells, had no effect. However, in STAT1 null (−/−) fibroblasts, little morphological change and apoptosis were observed in FAK alone transfected cells. Furthermore, these cells could undergo apoptosis when STAT1 was re-introduced with FAK.

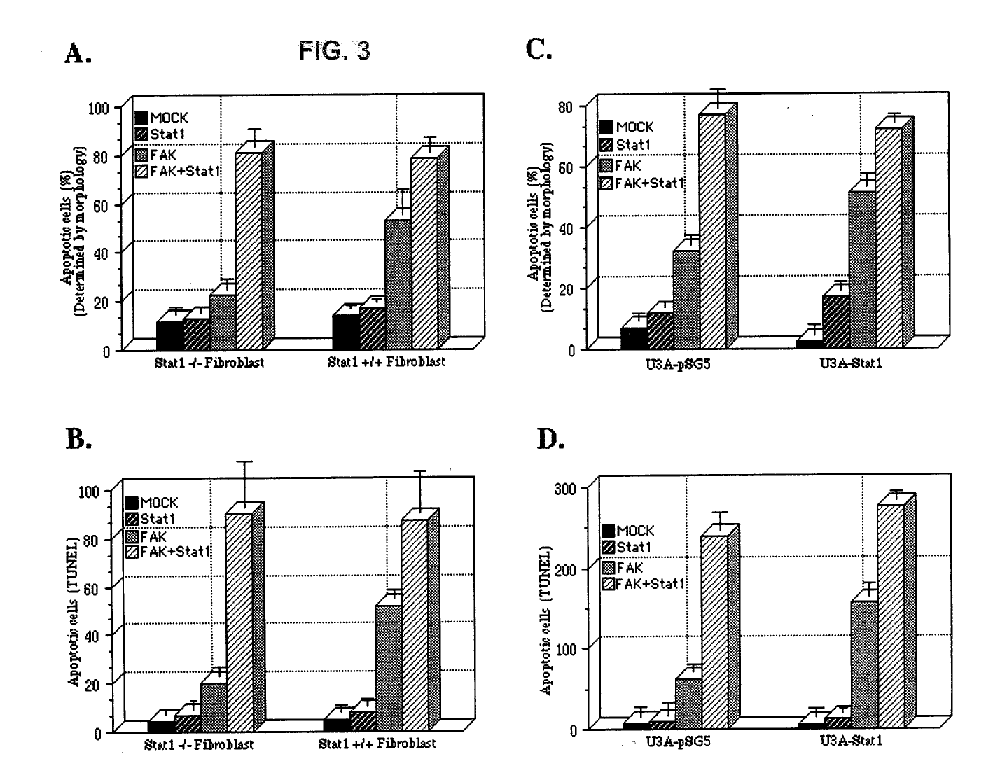

The results of quantitative measurement of apoptotic cells by the morphological examination (FIG. 3A), or measured by the TUNEL assay (FIG. 3B) were consistent. Please be noted that in the calculation, only transfected cells which were stained blue in the total STAT1 deficient or wild-type cells were measured. Similarly, the relative numbers of apoptotic cells observed in each field were counted and compared. The results were derived from three repeated experiments.

In addition to using these STAT1 null fibroblasts, U3A-pSG5 cell line was also used, which is STAT1 defective, and U3A-STAT1 cells, in which STAT1 has been stably reintroduced (Chin, et al., 1996), to further determine whether introduction of STAT1 to the STAT1 defective cells can confer FAK-induced apoptosis. As anticipated, transfection by FAK alone induced significantly more apoptotic cells in STAT1 positive cells than in STAT1 defective cells as determined by both cell morphology (FIG. 3C) and TUNEL assay (FIG. 3D). These results suggest that FAK-STAT1 activation is necessary for the induction of apoptosis in these transfectants.

Both Integrin Signaling and STAT1 are Necessary for Promotion of Apoptosis under Physiological Conditions Caused by Serum Withdrawal. Previous studies have indicated that a role of FAK is to prevent apoptosis under certain conditions. This might be due to the fact that FAK activates survival signals (RAS, PI3 kinase etc.) in parallel. Moreover, the cell culture media contain growth factors which provided additional survival signals. Thus the activation of STAT and the apoptosis signal might be negated or covered.

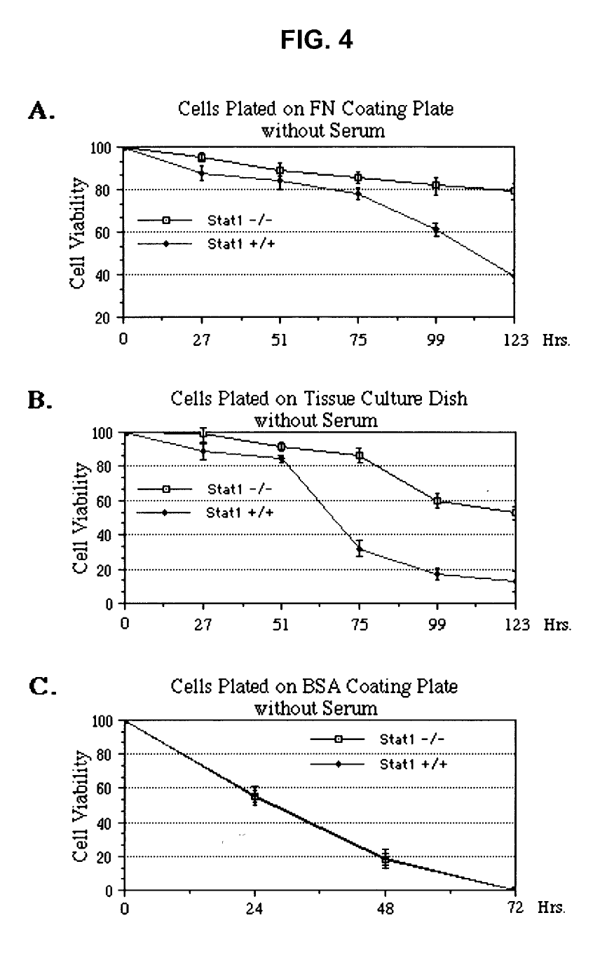

Embryonic fibroblasts, derived from either STAT1 null or wild type mice, were plated on fibronectin (FN) in a culture media containing no serum. If STAT1 contributes to the induction of apoptosis in response to the integrin-FAK signaling after cells are plated on fibronectin, then STAT1 wild type fibroblasts will undergo apoptosis faster than STAT1 null fibroblasts under this stringent condition. The results supported this hypothesis: STAT1 positive cells plated on FN or tissue culture dish (on which the cells are able to secrete matrix proteins) were dying significantly faster than those STAT1 null cells under the same condition (FIGS. 4A and 4B).

As an important control, these cells were also plated on bovine serum albumins (BSA), which does not activate integrin signaling. In contrast to the faster cell death rate for STAT1 wild type cells plated on FN (FIG. 4A), it was found that STAT1 null and wild type cells were dying at the same rate when they were plated on BSA (FIG. 4C).

These experiments have further shown that STAT1 protein can promote apoptosis, and this promotion of apoptosis through STAT1 is dependent on the integrin signaling which is triggered by adhesion to FN, but not to BSA. These results have further implicated that integrin-induced STAT activation can promote apoptosis under the physiological conditions when the survival signals are weakened, such as after serum or growth factor withdrawal. Moreover, no overexpression of STAT or FAK proteins was involved under the experimental conditions above.

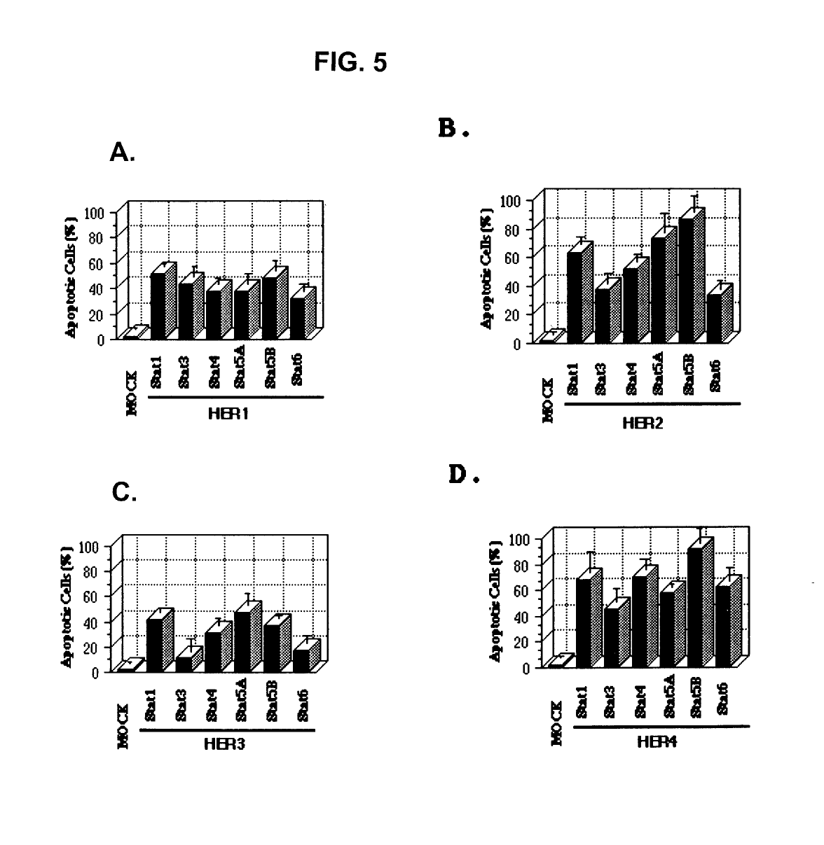

Induction of apoptosis by co-expression of the HER receptor family and STAT proteins. Similar to apoptosis induction through expressing FAK and STAT, co-expression of EGF receptor (HER-1) or each of other members of HER receptor family, with each member of STAT proteins (except STAT2) can cause induction of apoptosis (FIG. 5). Vectors expressing each member of the HER family and vectors expressing each member of STAT proteins were co-transfected into 293T cells. The apoptotic cells were identified by cell morphological changes and trypan blue exclusion. The results were derived from three repeated experiments. Joint actions of HER1 and each STAT proteins cause cell death (FIG. 5A); joint actions of HER2 and each STAT proteins cause cell death (FIG. 5B); joint actions of HER3 and each STAT proteins cause cell death (FIG. 5C); and, joint actions of HER4 and each STAT proteins cause cell death (FIG. 5D).

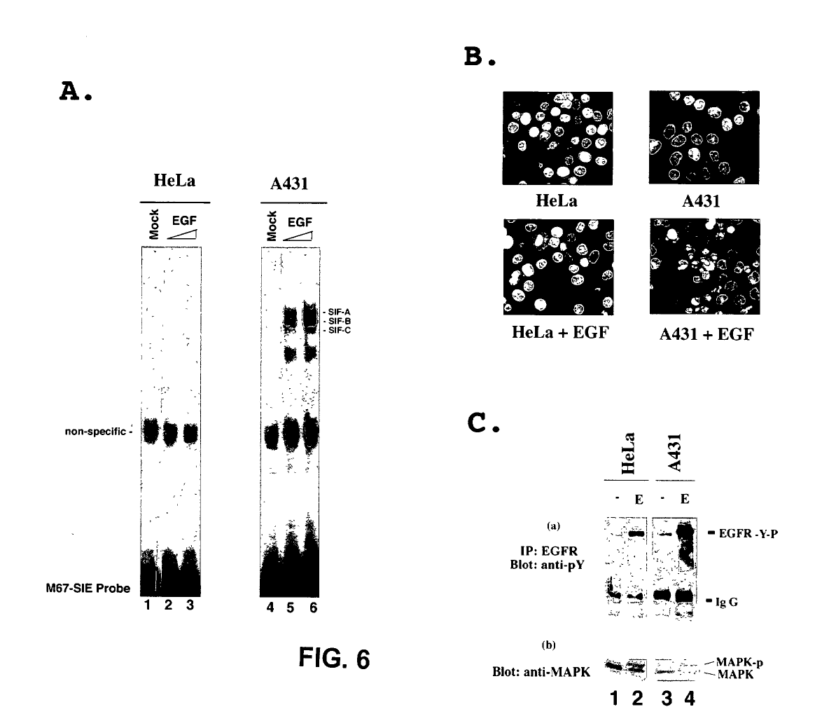

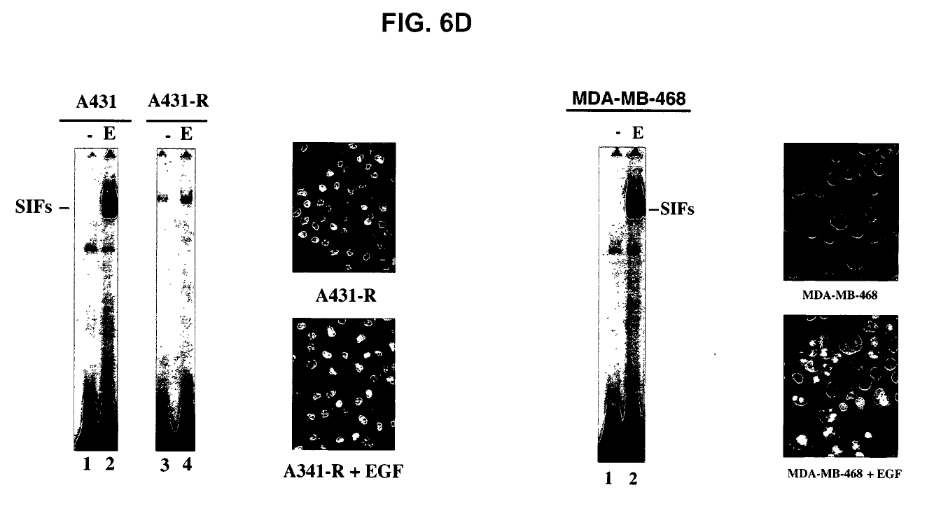

STAT activation induced by EGF causes apoptosis. Comparison of STAT activation in HeLa cells vs. A431 cells in response to EGF (FIG. 6A). Cells were treated with EGF for 30 min, and protein extracts were prepared. Electrophoretic mobility shift assays (EMSAs) using M67SIE as the probe showed that in A431 cells treated with EGF, DNA-bound STAT dimers were formed (SIF-A: STAT3 homodimer, SIF-C: STAT1 homodimer, and SIF-B: STAT1/STAT3 heterodimer). In contrast, no obvious STAT activities were detected in HeLa cells treated with EGF under the same conditions.

Apoptosis induction of these two cell lines correlates with STAT activation in response to EGF treatments (FIG. 6B). In A431 cells, EGF treatment induced cell apoptosis. In contrast, no apoptosis was observed in EGF-treated HeLa cells. The apoptotic cells were clearly identified by altered nuclear structure with condensed chromatin fragments seen under fluorescence microscopy after staining with fluorochrome bis-benzimide. EGF-induced apoptosis was further confirmed by DNA fragmentation assays (data not shown).

Since EGF elicited very different response in these two cell lines, EGF receptor autophosphorylation and MAP kinase activity in A431 and HeLa cells were examined (FIG. 6C). EGF treatment lead to EGF receptor autophosphorylation in both A431 and HeLa cells. The same protein extracts were also probed with anti-MAP kinase antibody in Western blot assays. MAP kinase (ERK-2) was phosphorylated (slowed mobility) and therefore activated after EGF treatment in both A431 and HeLa cells. These data indicate that failure of EGF to activate STAT proteins in HeLa cells was not due to an EGF receptor defect. The data also indicate that EGF-induced apoptosis in A431 cells was not due to inactivation of the Ras-MAP kinase pathway.

Additional evidence for the correlation between Receptor/PTK-STAT activation and apoptosis was obtained from the studies of MDA-MB-468 cells, a breast cancer cell line and A431-R, an A431 variant (FIG. 6D). It was found that STAT was not activated by EGF, consequently, no apoptosis was induced by EGF in A431-R cells. In contrast, in MDA-MB-468 cells, apoptotic cells were induced which was apparently caused by STAT activation (right panel).

ICE Expression Correlated with EGF-STAT activation and induced Apoptosis. Since the ICE protease family play important roles in apoptosis, the gene expression patterns of most members of these two apoptosis gene families were examined by Northern blot analysis (FIGS. 7A, 7B, and 7C). Among the genes tested, ICE (Caspase-1) expression was upregulated in a STAT-dependent manner. EGF induced ICE gene expression in both A431 and MDA-MB-468 cells, but not in HeLa cells which was correlated with STAT activation in these cells. In addition, in A431-R cells, which are defective in STAT activation, ICE mRNA expression was uninducible (data not shown).

To confirm that ICE induction at the protein level, Western blot analysis of whole-cell protein extracts from A431, HeLa, and MDA-MB-468 cells, which were treated with or without EGF, revealed that ICE protein levels increased following EGF treatments. A proteolytically cleaved form of ICE, p10, was clearly observed in A431 cells after EGF treatment. To obtain further evidence for the involvement of ICE in the EGF-induced apoptosis, it was examined whether ZVAD, an irreversible inhibitor of ICE family proteases can block EGF-induced apoptosis. ZVAD effectively blocked either EGF- or IFN-γ-induced apoptosis in cells tested.

Taking together these data, It was concluded that induction of ICE mRNA and protein are due to STAT activation in these cells, indicating that ICE protease may be involved in EGF-induced apoptosis.

Jak1 is necessary for induction of apoptosis in response to IFN-γ. Like many other cytokines, interferons may activate multiple pathways including the STAT and the Ras-MAP kinase pathways (David et al., 1995; Xia et al., 1996). Both ICE mRNA and apoptosis induction in JAK- and STAT-deficient cell lines in response to IFN-γ treatment were investigated.

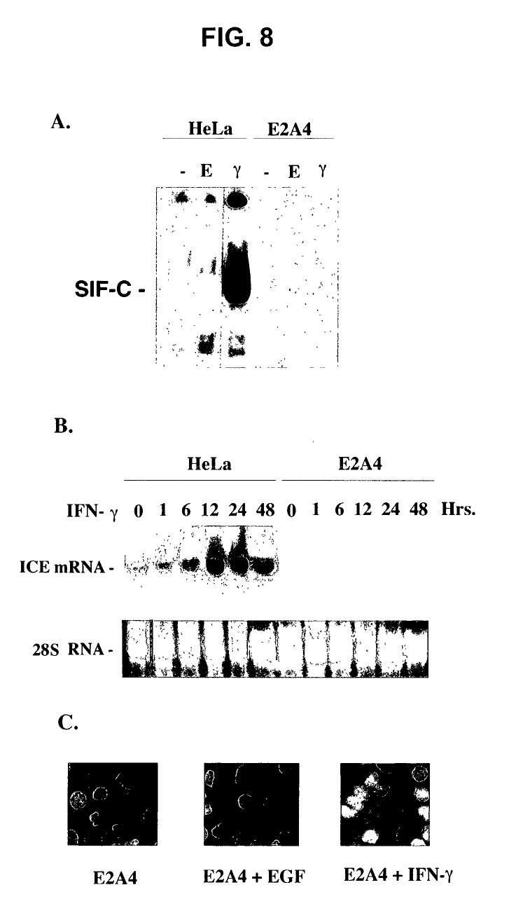

E2A4 is a JAK1 kinase-deficient cell line derived from HeLa cells (Loh et al., 1994). DNA binding activity of STAT activation was absent as determined by EMSAs in E2A4 cells (FIG. 8A). The strong induction of ICE mRNA normally seen upon IFN-γ treatment in the parental HeLa cells was completely abolished in this JAK1 deficient cell line (FIG. 8B). Moreover, bis-benzimide staining showed that E2A4 cells did not apoptose in the presence of IFN-γ (FIG. 8C). These results (A-C) suggest that JAK1 kinase is essential for the induction of both ICE expression and apoptosis by IFN-γ.

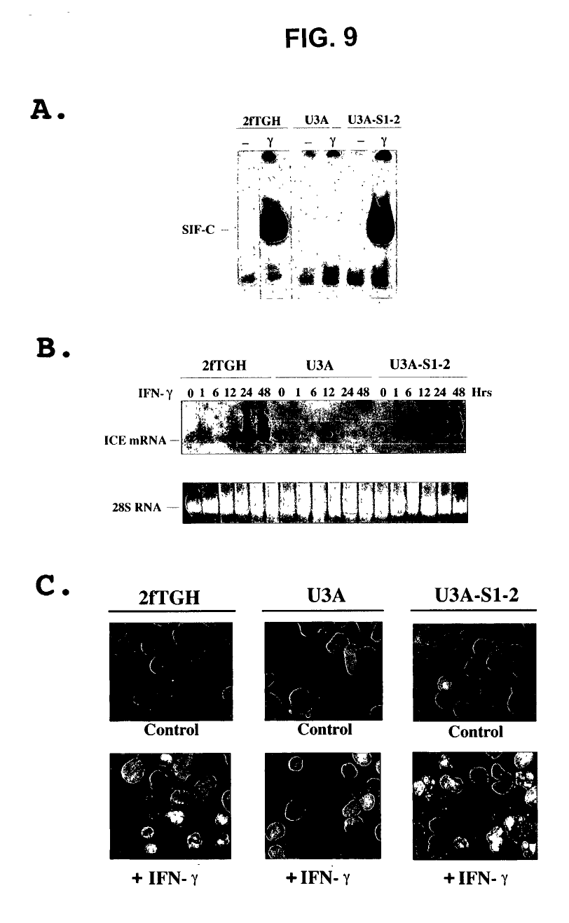

Apoptosis induction in response to IFN-γ. Analysis of apoptosis induction in U3A cells, a STAT1-defective cell line (McKendry et al., 1991). and the parental cell line 2fTGH, and STAT1 reintroduced U3A-S1-2 cells in response to IFN-γ. IFN-γ failed to activate STAT, induce ICE mRNA expression, or lead to apoptosis in U3A cells. In contrast, IFN-γ was able to induce STAT-DNA binding activity, ICE mRNA, and apoptosis in both the U3A parental cell line 2fTGH cells and the U3A-S1-2 cells, in which STAT1 had been reintroduced (FIGS. 9A and 9B). As a control, U3A cells that were stably transfected with the vector alone were unable to respond to IFN-γ (Chin et al., 1997).

Bis-benzimide staining of parental 2fTGH cells, STAT1 defective U3A, and STAT1 reintroduced U3A-S1-2 cells. The condensed and/or fragmented nuclei were observed in 2fTGH and U3A-S1-2, but not U3A cells treated with IFN-γ (FIG. 9C).

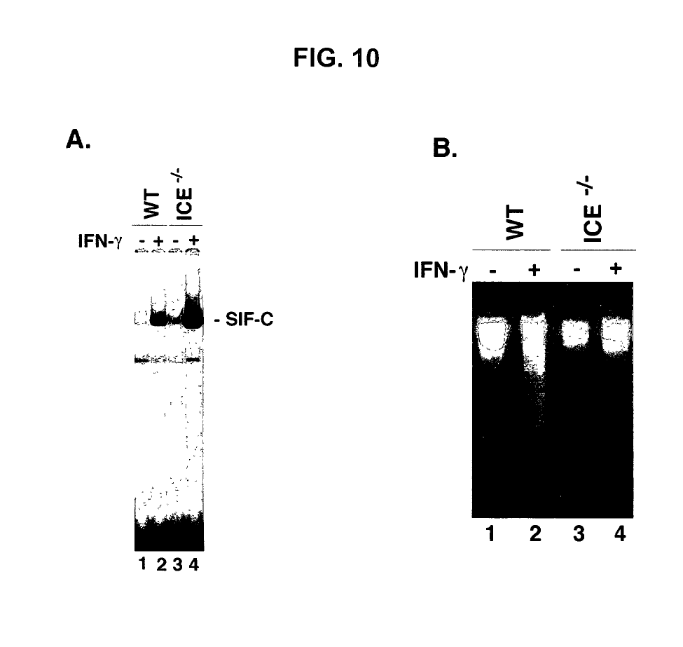

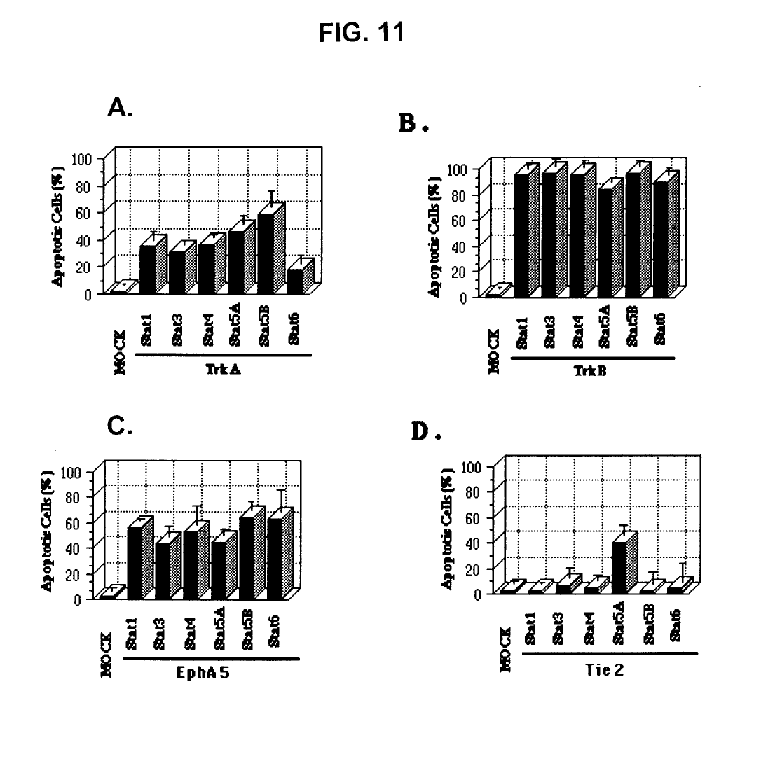

ICE Gene Is necessary for IFN-γ-Induced Apoptosis. To further confirm that ICE expression induced by IFN-γ is critical to provoke apoptosis, the primary spleen cells from ICE^{-/-} and ICE^{+/+} mice (Kuida et al., 1995) were isolated and their responses to IFN-γ treatment were compared. Although STAT1 can be activated by IFN-γ in both ICE^{-/-} and ICE^{+/+} cells (FIG. 10A), IFN-γ-induced DNA fragmentation was significantly reduced in ICE^{-/-} cells compared with that in ICE^{+/+} cells (FIG. 10B). The cell viability assays (trypan blue exclusion) showed that IFN-γ triggered much more apoptosis in ICE^{+/+} cells than in ICE^{-/-} cells, in a dose-dependent manner (Chin et al., 1997). Thus, ICE expression plays an important role in IFN-γ-induced apoptosis.

A General Pathway to Induction of Apoptosis through the Joint Actions of a variety of different Receptor/PTKs and STATSs. Besides the above well-characterized examples of apoptosis induction through activation of STAT proteins by the family of EGF receptor kinases, FAK, and Jak kinases, apoptosis induction through joint actions of a variety of Receptor/PTKs with each of the STAT proteins after they are pairly transfected into 293T cells, have been further analyzed:

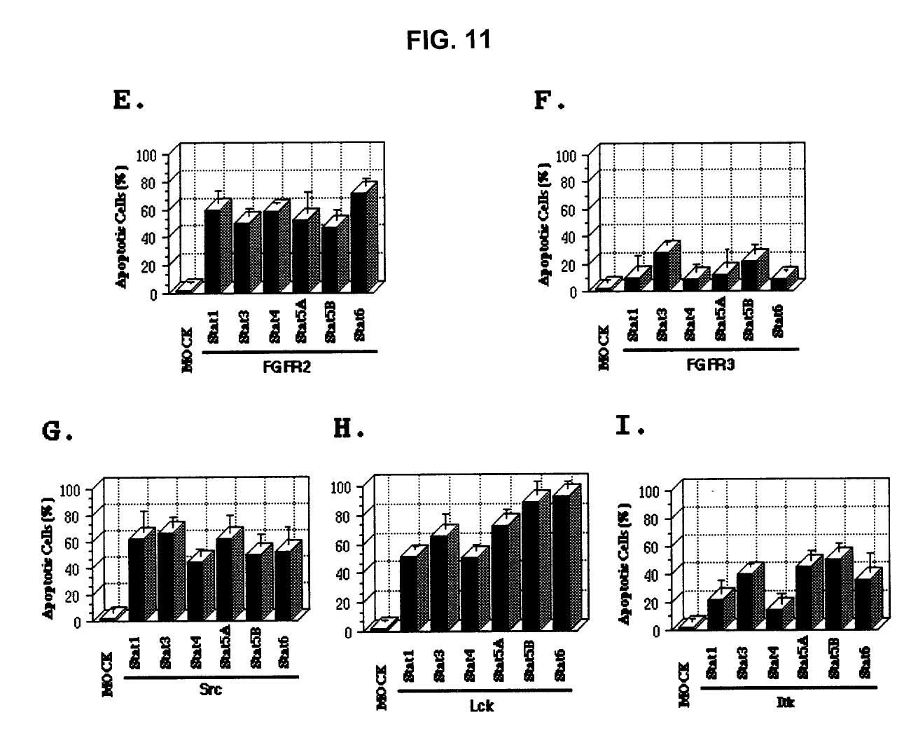

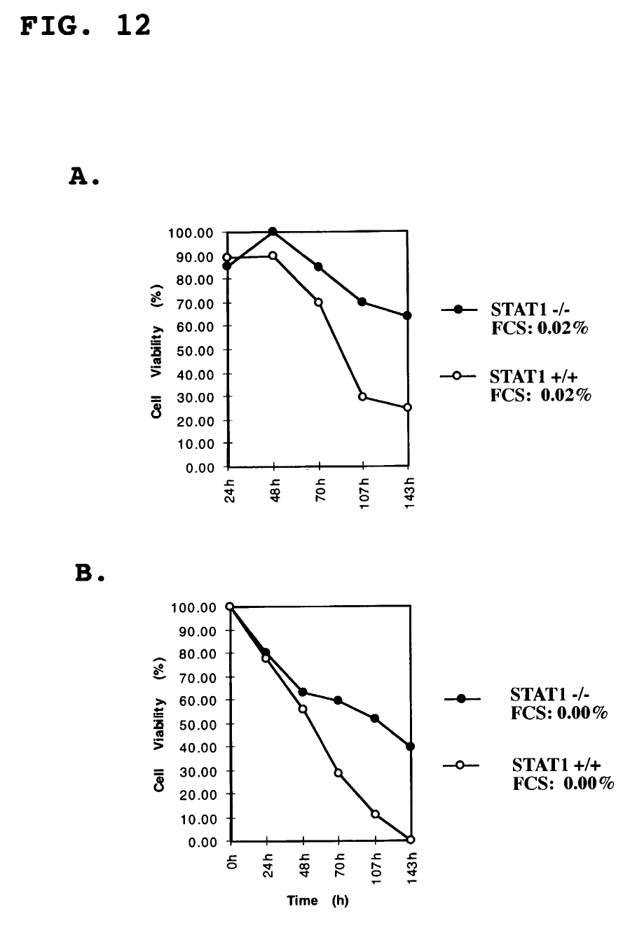

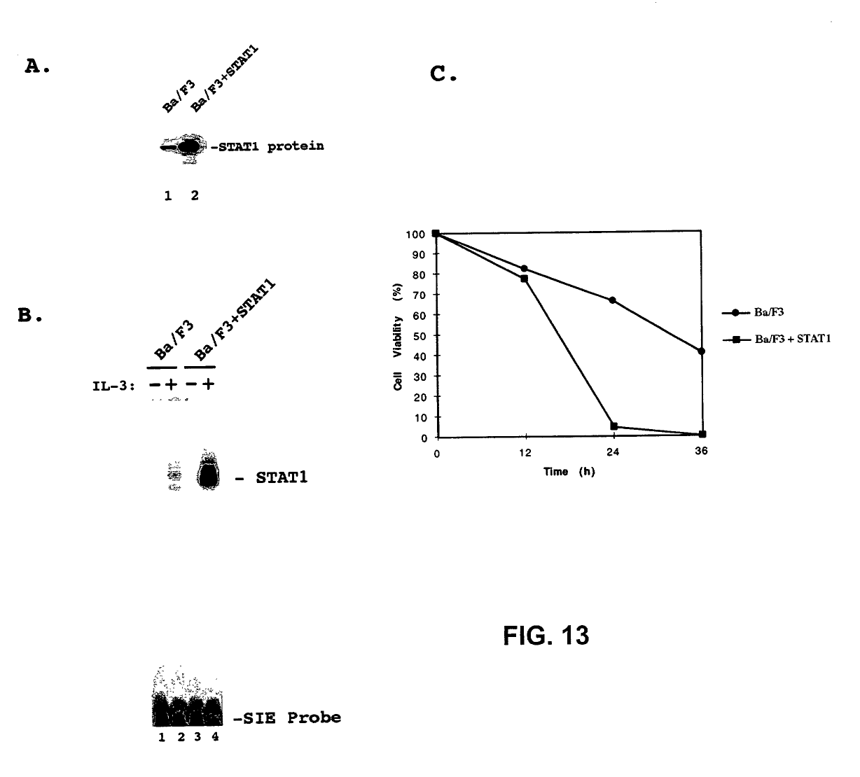

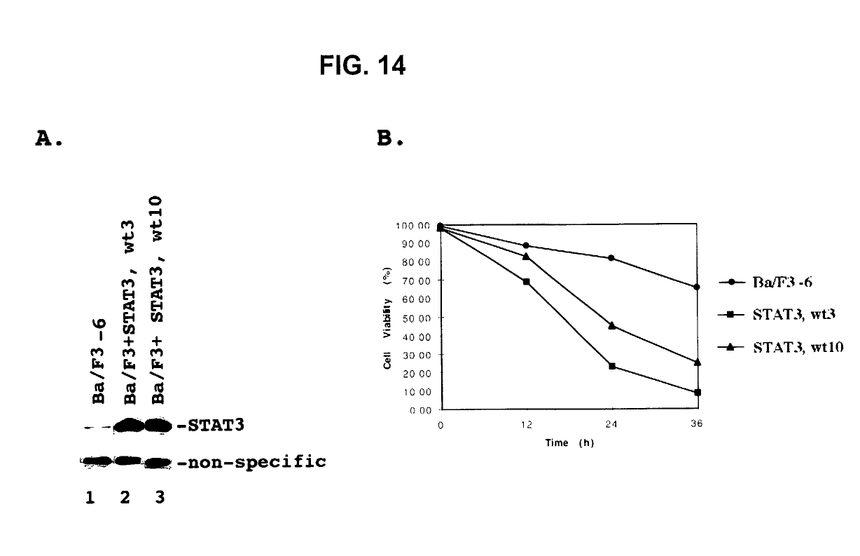

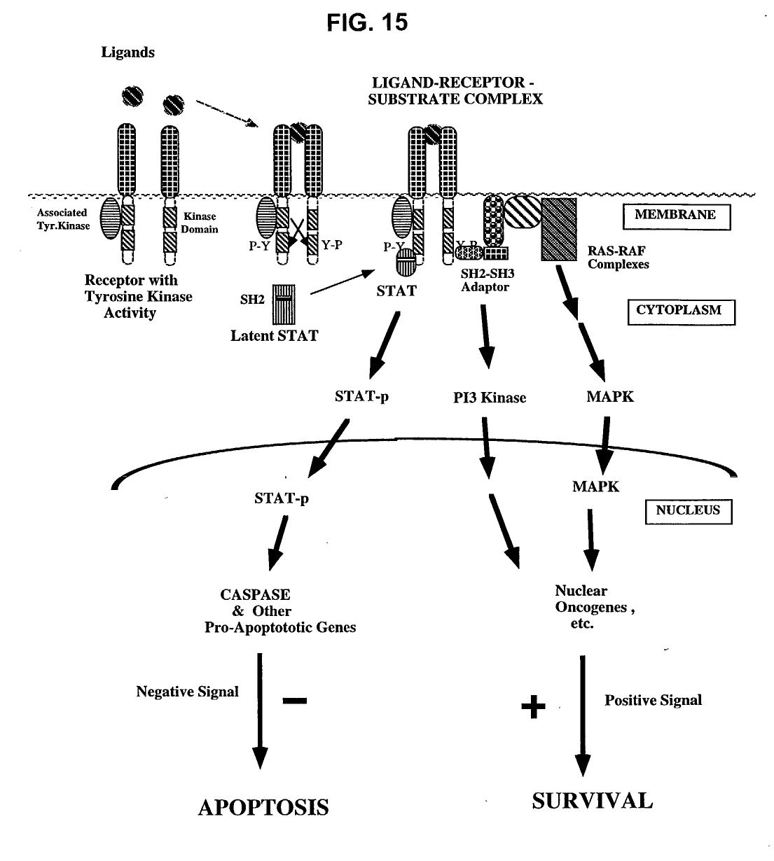

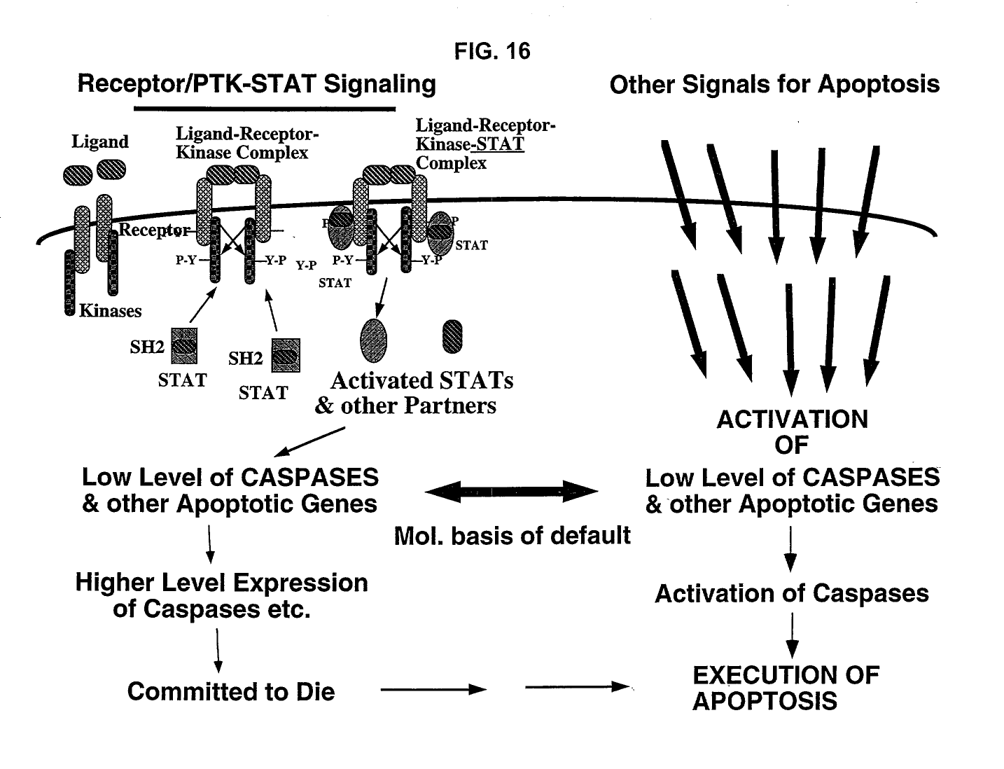

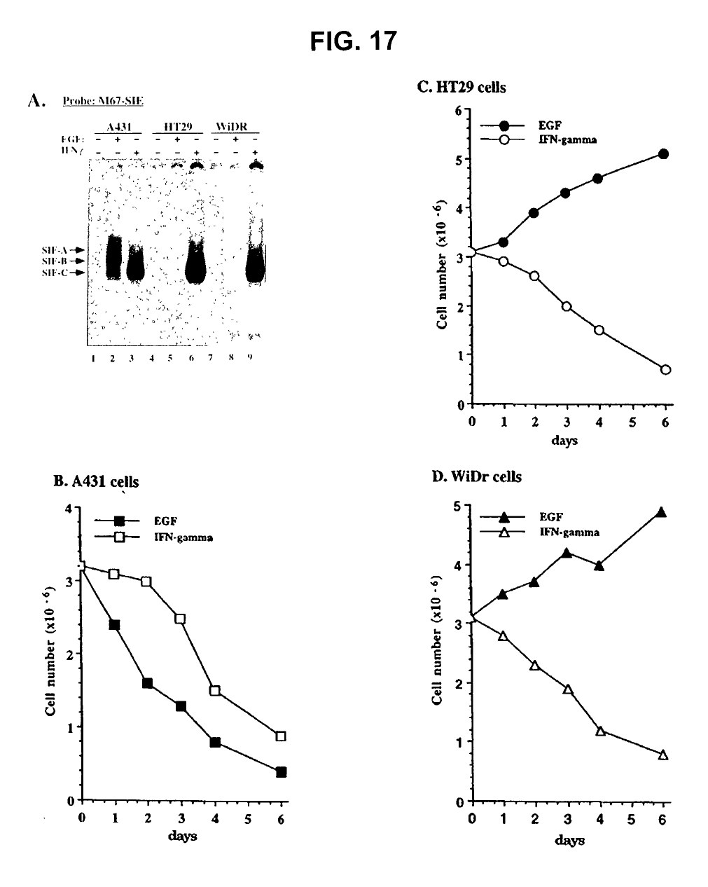

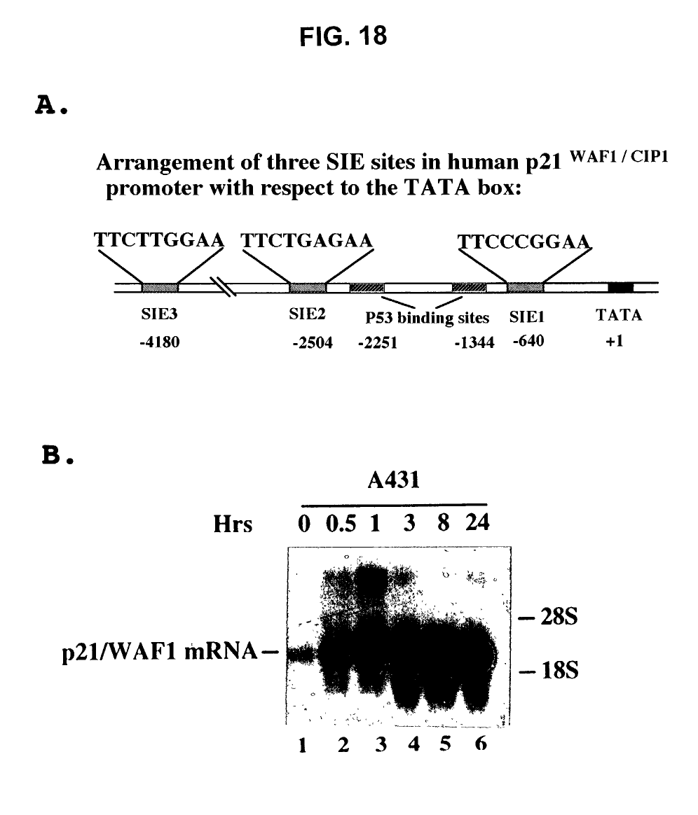

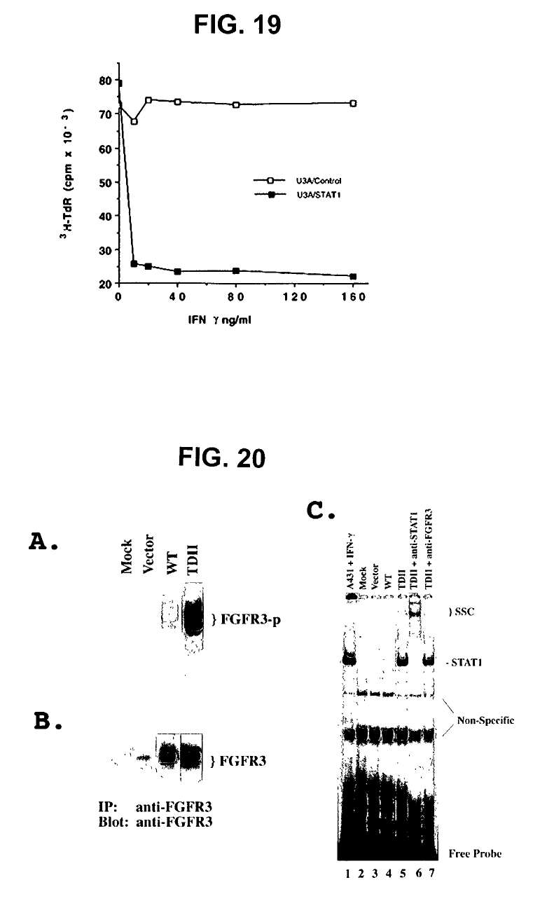

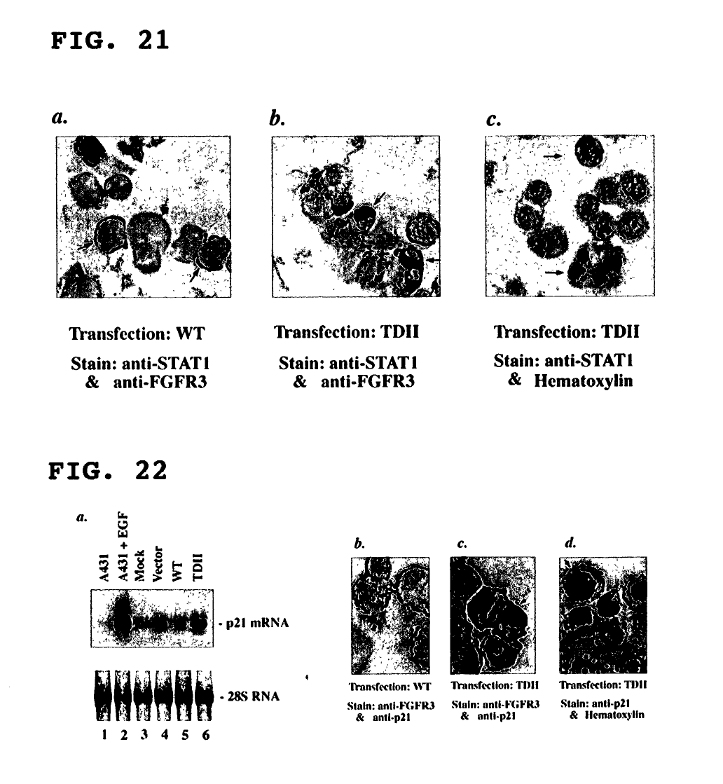

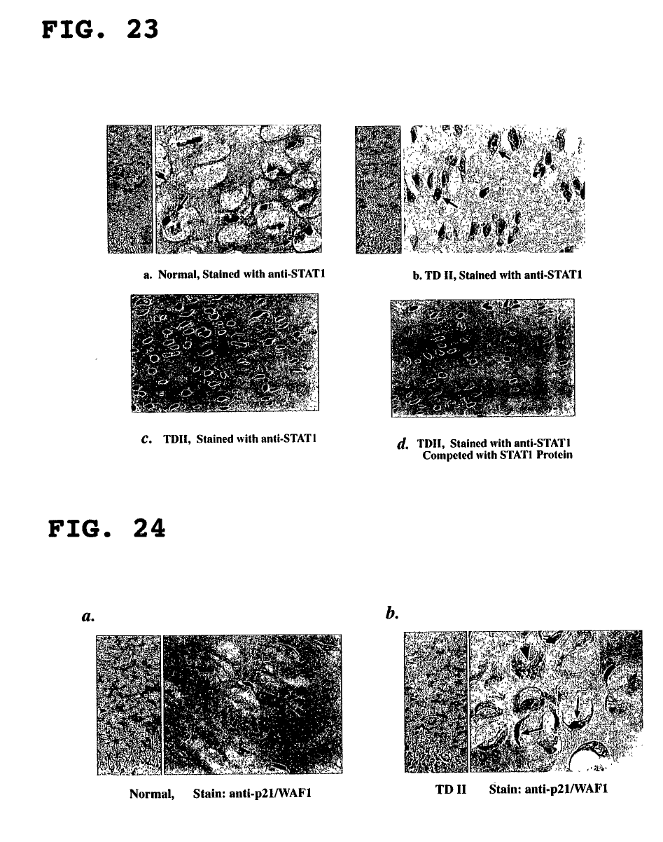

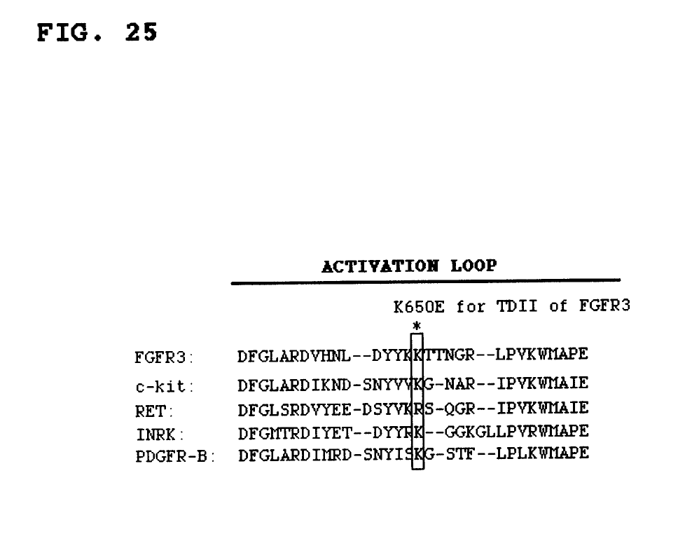

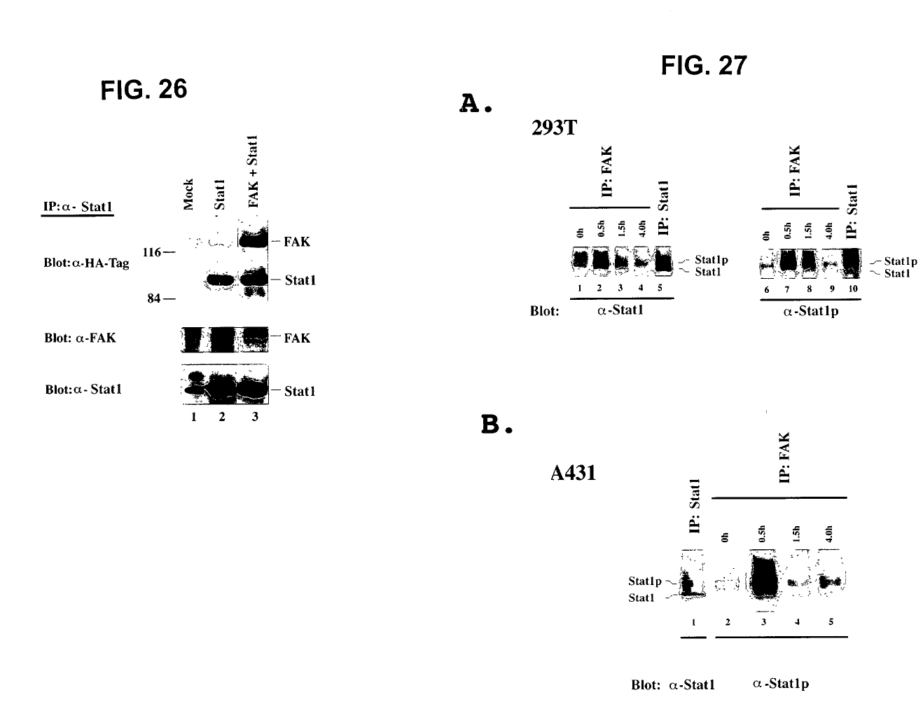

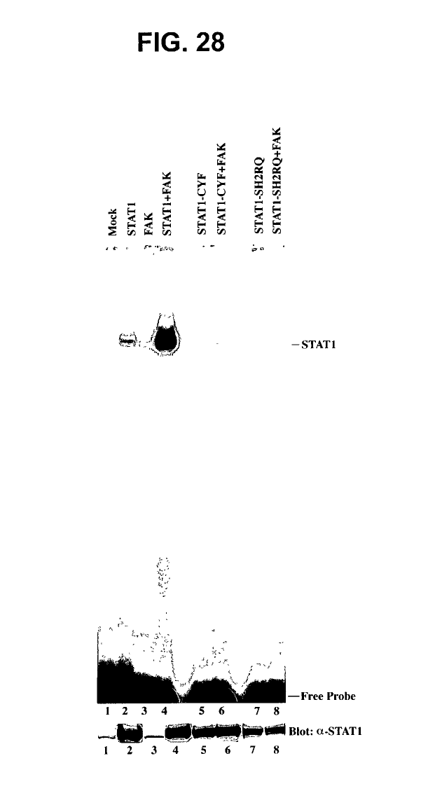

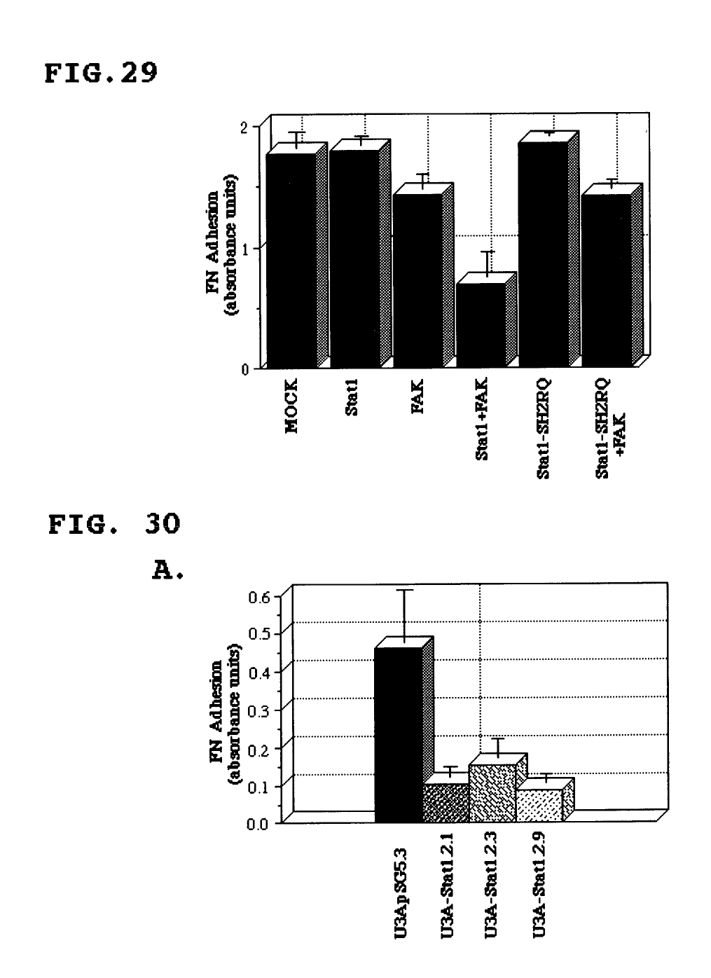

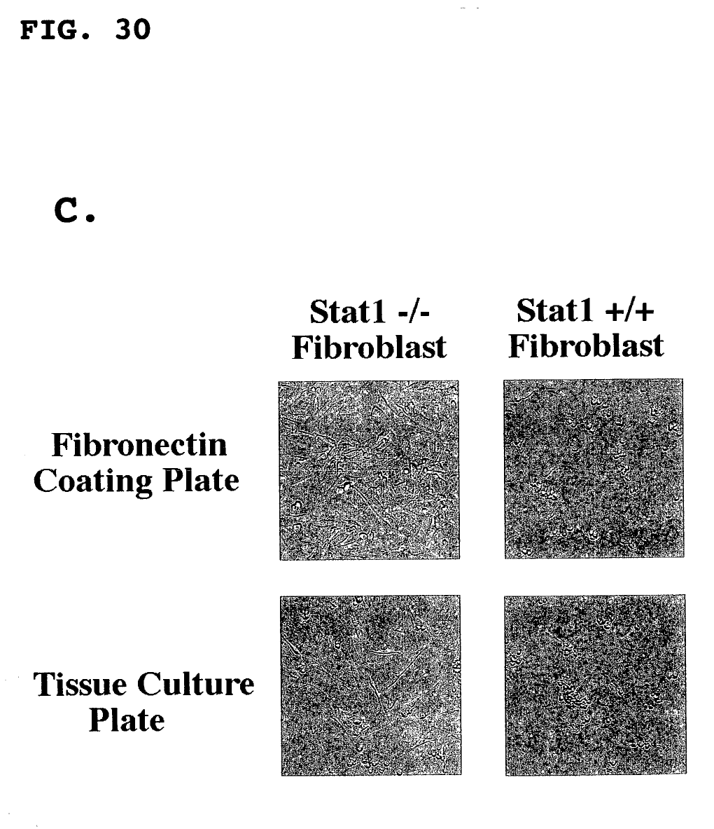

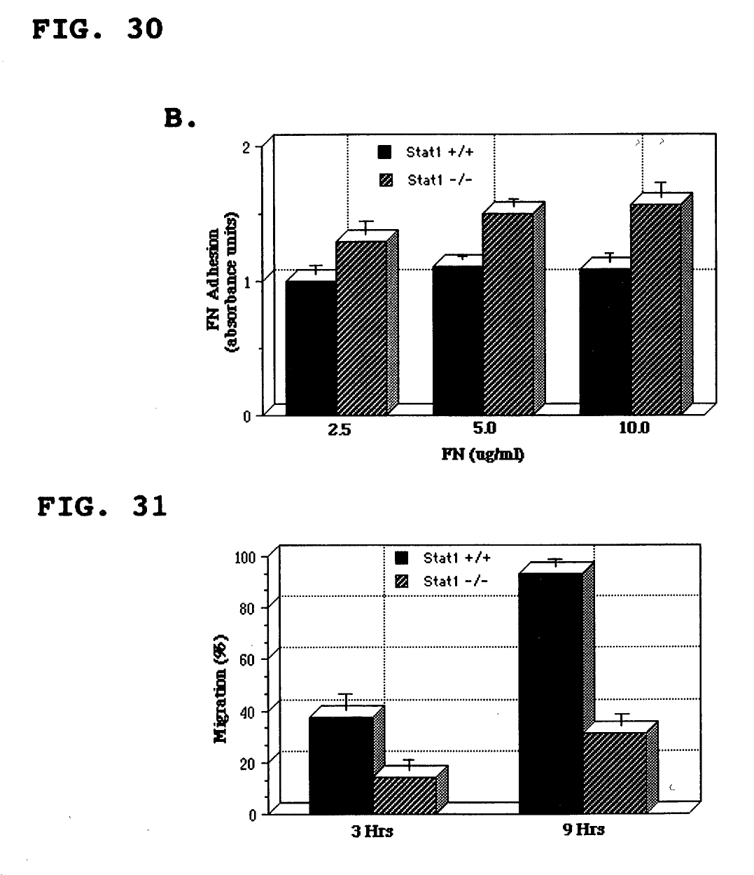

FIG. 11A shows apoptosis induction through joint actions of TrkA, a nerve trophin receptor, and each of the STAT proteins;

FIG. 11B shows apoptosis induction through joint actions of TrkB, a nerve trophin receptor, and each of the STAT proteins;

FIG. 11C shows apoptosis induction through joint actions of a EPH protein, a nerve trophin receptor involved in neuron differentiation, and each of the STAT proteins;

FIG. 11D shows apoptosis induction through joint actions of Tie2, a receptor involved in angiogenesis and early development etc., and each of the STAT proteins; STAT5A was especially active in causing apoptosis;

FIG. 11E shows apoptosis induction through joint actions of FGFR2, a receptor involved in development and angiogenesis etc., and each of the STAT proteins;

FIG. 11F shows apoptosis induction through joint actions of FGFR3, a receptor involved in development and angiogenesis etc., and each of the STAT proteins;

FIG. 11G shows apoptosis induction through joint actions of Src, a cytoplasmic tyrosin kinase involved in bone development and tumor transformation etc., and each of the STAT proteins;

FIG. 11H shows apoptosis induction through joint actions of Lck, a cytoplasmic tyrosin kinase involved in lymphocytes development and function etc., and each of the STAT proteins;

FIG. 11I shows apoptosis induction through joint actions of Itk, a cytoplasmic tyrosin kinase involved in lymphocytes development and function etc., and each of the STAT proteins.

The results of quantitative measurement of apoptotic cells are obtained by the morphological examination, and by the trypan-blue exclusion assay, and only transfected cells were accounted which were stained blue dye to co-transfection with a vector that expressed b-galactosidase, thus transfectants could be specifically recognized by the blue color after X-gal staining. The mock was the vector alone transfected cells.

The STAT proteins control the apoptosis induction by default after growth factor withdrawal. Cultured mammalian cells will die through apoptosis when the necessary cytokines, growth factors or serum are deprived. This induction of apoptosis after growth factor or serum withdrawal has been believed to be due to a “default” mechanism (Raff, 1992). According to this notion, cells can only survive when growth factors are provided to suppress this mechanism to die. It is not known what is the molecular basis of this default mechanism. It is unclear either whether this induction of apoptosis by default can be regulated and affected by signaling pathways. Nothing is known about the mediators that carry out this apoptosis by default.

The importance of cell death induced by growth factor deprivation, or by default is not only limited in cultured cells. This kind of cell death occurs commonly during certain critical developmental stages. It is required for organogenesis and maintenance of homeostasis of whole body. Furthermore, a variety of degenerative diseases should be caused by apoptosis through reduction or deprivation of survival factors, an event resembling cell death triggered by growth factor withdrawal in culture cells (Thompson, 1995). It was shown that STAT proteins control the induction of apoptosis caused by growth factor withdrawal. In other words, induction of apoptosis by default after survival factor deprivation, which is a mechanism involving many kinds of degenerative diseases and developmental processes, can be controlled by STAT-regulated expression of apoptosis genes.

Expression of the STAT1 protein in mouse embryonic fibroblasts promotes apoptosis by default after serum withdrawal while deficiency of STAT1 protein reduces apoptosis after serum withdrawal. The comparison of apoptosis induction after serum reduced to 0.02%. STAT1 deficient cells undergo apoptosis slower that STAT1 positive cells under these conditions (FIG. 12A). The comparison of apoptosis induction after serum withdrawal (0.0%). STAT1 deficient cells undergo apoptosis slower that STAT1 positive cells under these conditions (FIG. 12B).

Expression of the STAT1 protein in Ba/F3, a cell line derived from pro-B cells, promotes apoptosis after serum or growth factor (IL-3) withdrawal. STAT1 protein expression is higher in a Ba/F3+STAT1 cell clone that expresses exogenous STAT1 than its parental cells (FIG. 13A). STAT1 SIE-DNA activities were higher in the Ba/F3+STAT1 cells than that of the parental Ba/F3 cells (FIG. 13B). Higher activities of STAT1 in Ba/F3+STAT1 cells cause faster cell death after serum withdrawal (FIG. 13C).

Expression of the STAT3 protein in Ba/F3, mouse embryonic fibroblasts promotes apoptosis after serum withdrawal. STAT3 protein expression is higher in two independent cell clones of Ba/F3+STAT3 (STAT3, wt3, and STAT3, wt10), that express exogenous STAT3, than its parental Ba/F3 cells (FIG. 14A). Higher expression of STAT3 protein in these two clonal cells cause faster cell death after serum withdrawal (FIG. 14B).

Discussion

In this Example, the present inventors have discovered, for the first time, new methods and compositions to regulate and modulate induction of apoptosis. They have shown the Receptor/PTK-STAT signaling pathway is crucial in the induction of apoptosis. This finding contrasts with the general concept that protein tyrosine kinase-activating cytokines usually act to stimulate growth and survival factors in a cell. However, the present inventors' finding supports the notion that many cytokines may have dual functions in cell growth control, generating either positive or negative growth signals depending on the cell types. Activation of STAT is one of these negative signals induced by receptor-associated tyrosine kinases.

As shown in FIG. 15, a negative and positive signaling model is proposed to explain the molecular basis responsible for the dual functions of cytokines. It is proposed that a cytokine, by binding to its receptor, can turn on at least two separate signaling pathways: activation of the Ras-MAP kinase pathway (or other pathways such as PI3 kinase pathway) for cell growth/survival and activation of the STAT pathway for cell arrest/death. The intracellular homeostasis requires a balance between growth/survival and arrest/death signaling events. Different cells may have different dynamic states and hence different phenotypic outputs. In HeLa cells, the STAT pathway is not very active, EGF mainly triggers the MAP kinase pathway, so cells proliferate and survive. While in A431 or MDA-MB-468 cells, the STAT pathway is more sensitive in response to EGF, the caspase can be induced and cells arrest and die. Therefore, EGF can activate a negative signaling pathway through STAT activation and expression of caspases.

Therefore, the polypeptide ligand-activated Receptor/PTK signaling, could not only transduce the proliferative and surviving signals, as most commonly observed, but also, in some conditions, generate anti-proliferative and cell death signals which are mediated by the STAT proteins. This conclusion may provide a solid explanation for the dual functions of many cytokines and growth factors. The discoveries made in this invention showing that the Receptor/PTK-STAT signaling can lead to apoptosis which will provide methods and compositions for finding agents to interfere apoptosis during development and apoptosis during development of a variety of mammalian degenerative diseases which are triggered by survival factor deprivation.

Several important issues concerning the conclusions in this invention should be further clarified here. First, the greatly reduced apoptotic response in ICE^{-/-} cells (see above) constitutes definitive evidence for the requirement for ICE in IFN-γ-induced cell death. While ICE expression is involved in the EGF- and IFN-γ-induced apoptosis as demonstrated here, it could be a general mechanism that regulation of caspases by each of the STAT proteins is the molecular basis in many other conditions, such as in cell matrix-related, in particular, integrin-induced apoptosis shown above. Additionally, caspase-1 is the first, but may not be the only caspase That can be regulated by the Receptor/PTK-STAT signaling. Other members of caspases may also be targets of regulation. For example, ICH-1/caspase 2 could also be up-regulated by STAT activation (data not shown). In some cells, BAX and FAS may also be up-regulated by the STAT pathway (inventors' unpublished results). Therefore, it must have multiple target genes in STAT-mediated apoptosis induction. Therefore the regulation of caspases by the STAT pathway can be regarded as the first model system.

Furthermore, some diseases resulting from inappropriate cell arrest and apoptosis may be due to an overactive Receptor/PTK-STAT pathway. It has been shown that in this invention that a variety of Receptor/PTKs, activate different STAT proteins and induce apoptosis. For example, the Receptor/PTK-STAT can be strongly activated and the induction of caspases is high after cytokine treatment. Therefore, the survival signals from Receptor/PTK are overcome by the effects of STAT activation and upregulated caspases, causing apoptosis.

Importantly, The Receptor/PTK-STAT signaling pathway and its regulated gene expression may provide a molecular mechanism of the apoptosis induction by DEFAULT. There must be certain mediators or gene products to carry out the death execution after survival factor deprivation, based on the view that apoptosis is an active process. It is known that for different types of cells there are significant differences in their sensitivities or thresholds for apoptosis induction by default (Thompson, 1995). The Receptor/PTK-STAT pathway can mediate induction of apoptosis through the regulation of caspases and/or other pro-apoptotic proteins. However, in normal cell culture conditions without large amount of cytokines, STAT proteins may only be activated at a lower level (possibly due to growth factors in the serum supplied, or matrix/integrin interactions etc.). Coordinately, a lower and constitutive level of caspases or other pro-apoptotic gene expression is maintained which is not strong enough to overcome the survival signals. Thus these cells survive. However, when the survival signals are deprived, such as when growth factors are withdrawal, the balance between death signals (STAT and caspase activities etc.) and survival signals may shift towards death. Thus apoptosis may occur. Evidence was presented in this invention to support this important conclusion. For example, it is shown above that STAT activation through integrin signaling could promote apoptosis when serum were deprived. Additionally, since caspase activities are required for most, if not all, apoptosis execution, STAT-regulated caspase expression should also affect many kinds apoptosis induction through different signaling pathways, which may include apoptosis induction caused by radiation, TNF/FAS, Myc expression, and many other apoptosis-inducing agents. This conclusion is shown in the FIG. 16.

Thus, according to principles presented in FIG. 16, the hard-wired, intrinsic executioner or mediator (THE DEFAULT) of apoptosis is the low level of STAT activities and STAT-regulated caspase or other pro-apoptotic protein activities. The differences in the thresholds for induction of apoptosis may be determined by the different levels of STAT and STAT-regulated caspase or other pro-apoptotic protein activities. This conclusion may be correct for many situations in which apoptosis is induced due to weakened survival signals. These are significant and fundamental issues, since apoptosis induction by this default mechanism after survival signal withdrawal may be responsible for many important situations of apoptosis induction during stages of development or in the pathogenesis of auto immune disorders, leukemia, and some degenerative diseases, and for many other types of “spontaneous” apoptosis. Moreover, although some caspases such as CPP32/caspase-3 may not be regulated by STAT proteins, its enzymatic activities may be activated by other caspases, such as caspase-1. It has been shown most STAT proteins can well play a role in induction of apoptosis (see Figures above). The possible redundant functions of different members of STATs and caspases may provide an explanation to the observation that a single null mutation of one member of STATs or caspases may not cause significant defects in development (Durbin et al., 1996; Kuida et al., 1995).

In summary, Receptor/PTK-STAT activation is a broad molecular signal mediating induction of apoptosis, and modulating Receptor/PTK-STAT activities can provide an important diagnostic and therapeutical tools and treatment methods for a variety of apoptosis-related diseases.

### EXAMPLE 2

Many growth factors and cytokines, such as EGF, PDGF, FGF, IL-3 and IL-6 etc., have been shown to play critical roles in cell proliferation, differentiation and development. Moreover, many mammalian diseases are associated with abnormal functions of growth factors. It is reasonable to speculate that as major functional mediators of these cytokines, STAT proteins may be involved in regulation of cell proliferation, development and in pathogenesis of some developmental disorders. However, it has been unclear until recently whether STAT proteins play any decisive roles in these important biological processes.

In the following studies, evidence was presented showing that STAT activation will induce immediate gene expression of CDK inhibitor p21, which may play key roles in cell growth arrest in response to cytokines and also may be involved in the regulation of some critical developmental steps. In particular, it was shown that growth defects in the bone development observed in mutant FGFR3 patients are probably caused by abnormal functions of Receptor/PTK-STAT signal transduction and induction of CDK inhibitor p21/WAF1. Thus, it was discovered that the Receptor/PTK-STAT pathway may play a decisive role in the negative regulation of cell growth.

Materials and Methods

Site-directed mutagenesis and plasmid construction Chameleon Double-Stranded, Site-Directed Mutagenesis Kit (Stratagene) was used to engineer the TDII type mutation on a mouse FGFR3 expression vector pMo/mFR3/SV (Ornitz et al. (1992)). An Oligonucleotide 5′-GGACTACTACAAGGAGACCACAAACGGCCGGCTACC-3′ (K644E in mouse) (SEQ ID NO. 2) and the Afl III primer from the manufacturer were used for the reaction. The successful mutagenesis was verified by sequencing. The mutated and wild-type cDNAs cloned into pEF-BOS vector were used for transfections otherwise mentioned (Muzushima et al. (1990).

Immunocytochemistry and immunohistochemistry. Two days after the transfection, 293T cells were harvested and smeared on glass slides. Cells were fixed and permeabilized in cold methanol/acetone (1:1) solution at −20 C. for 10 minutes, followed by incubation with normal horse or goat serum to block the nonspecific binding sites. Paraffin sections were de-paraffinized by the successive treatment with xylene, 95% ethanol and water. After treatment with FICIN (Zymed) enzyme to retrieve antigens, sections were incubated in 2.0% hydrogen peroxide and 0.1% sodium azide in methanol for 10 minutes to inactivate endogenous peroxidase. Antibodies against STAT1 (monoclonal for the cultured cells and polyclonal for the tissue sections, Transduction Laboratory), p21^{WAF1/CIP1} (Santa-Cruz), and FGFR3 (Santa-Cruz) and Vectastain Elite ABC Kit (Vector Laboratory) were used to stain the cells. 3,3′-Diaminobenzidine (DAB) with or without nickel chloride were used as substrate of peroxidase to give a black or brown color, respectively.

Northern blotting. Total RNAs (5 mg) from 293T cells transfected with the FGFR3 expression vectors were subjected to Northern blot analysis with the p21 cDNA used as a probe as previously described (Chin et al., 1996).

Growth assays. Twenty-four hours after the transfection, 293T cells were replated into 24-well culture plates at a density of 1.5×10⁴ cells/well and cultured for additional 24 hours. Then the cells were labeled with [³H]-thymidine (1.5 mCi/ml) for 4 hours, followed by washing with PBS twice, and in ice-cold 10% trichloroacetic acid three times. The incorporated radioactivity was extracted by incubation in 3% perchloric acid at 95 C. for 40 minutes and measured by liquid scintillation. Clonogenic assay was performed with 293T cells after the transient transfection as previously described (Chin (1996)), except that the concentration of the cells was reduced to 5×10³ cells per plate. Colonies were counted 10 days after plating.

Results

STAT Activation Induced by EGF and IFN-γ is Correlated with Cell Growth Arrest. Using M67-SIE as the probe in an EMSA, many cell lines were analyzed for STAT activation in response to EGF and found no or very poor STAT activation by EGF in most cells except A431 cells. The results from two representative cell lines, HT29 and WiDr, which are derived from human colon adenocarcinoma, are shown in FIG. 17A. In contrast to A431 cells in which STAT proteins (SIF-A: STAT3, SIF-C: STAT1, SIF-B:STAT1 and STAT3 heterodimer) were activated and cell growth was inhibited in response to EGF treatment, no detectable STAT activation was observed after EGF treatment of HT29 and WiDr cells, and these cells grew normally in the presence of EGF (FIGS. 17C and 17D). However, all these cells, including A431, were responsive to IFN-γ, producing activated STAT1 (SIF-C) as shown above. As expected, growth of all these cells was inhibited by IFN-γ treatment (FIGS. 17B, 17C and 17D). Additionally, results from ³H-thymidine incorporation assay were consistent with the growth curves (data not shown).

These results suggest that activation of STATs, STAT1 in particular, in A431 cells by EGF, as well as STAT1 activation in all these cells by IFN-γ, might be a key event leading to the inhibition of cell growth.

The p21/WAF1 Expression by STATs in Response to EGF and IFN-γ. Vogelstein and his colleagues have cloned the promoter region of the p21 gene and mapped the p53 regulatory sites (El-Deiry et al., 1995).

After a careful examination of this promoter, It was found that there are three sequences in this promoter that contain potential STAT interacting site (SIE) which contain a palindrome sequence TTC (N3) GAA (see Chin et al., 1996) (FIG. 18A). It has been further shown that these p21-SIEs are regulatory sites of STAT proteins (see Chin et al., 1996), raising the possibility that the p21 gene may be up-regulated by STAT proteins.

It has been found that p21 Gene Expression is Correlated with STAT Activation and Growth Suppression in Response to EGF and IFN-γ. One example is shown in which p21 mRNA was induced after EGF treatment (FIG. 18B). These SIE sites are possible targets for agents that block STAT-DNA interaction.

STAT1 is Essential for Induced Cell Growth Arrest. IF STAT1 is essential for growth inhibition, STAT1 deficient U3A cells will not be inhibited by IFN-γ. On the other hand, reintroducing functional STAT1 protein into U3A cells may restore its responsiveness, including cell growth arrest, to IFN-γ. U3A cells were restored by stable transfection with a STAT1 a expression vector pSG91 (Fu and Zhang, 1993). These STAT1a expressing U3A cells were named as U3A/STAT1 a. After these initial analyses, the rates of DNA synthesis by ³H-thymidine incorporation in U3A/Control and U3A/STAT1a cells in response to IFN-γ treatment were compared (FIG. 19).

As shown in above, U3A/Control cells which were deficient in STAT1 were not inhibited by IFN-γ even at high doses of IFN-γ, whereas the U3A/STAT1 cells were dramatically inhibited by IFN-γ at relatively low doses of IFN-γ. To further analyze the role of STAT1 in growth suppression, It was also found that U3A/STAT1a cells were greatly inhibited by IFN-γ for its ability to form colonies (about four fold fewer colonies) as compared with U3A/Control cells that were not affected by IFN-γ (Chin et al., 1996). These results have convincingly demonstrated that STAT1 was essential for the growth inhibition induced by IFN-γ in these cells. Consistently, p21 mRNA was expressed and induced at a much higher level in U3A/STAT1a than in U3A/Control cells (data not shown). Similarly, It was further shown that STAT activation is also required for EGF induced p21 induction and cell growth arrest in A431 cells and other EGF inhibitory cells such as breast cancer MDA-MB-468 cells (data not shown). In summary of the above results, it has been clearly shown that the activation of STAT proteins is essential for growth suppression in response to IFN-γ and EGF, probably through induction of CDK inhibitors like p21.

STAT1 Activation induced by expression of a mutant TDII FCTFR3 receptor. It has been shown that the FGFR3 mutants have a gain-of-function nature leading to the abnormal and excessive repression of bone growth. Although FGF is known for its proliferative effects, the phenotypes of mutant FGFR3 related disorders suggest that the biological effect of these FGFR3 mutants at the cellular level is inhibition or retardation of chondrocyte proliferation in the cartilaginous growth plates. For example, the achondroplasia class of chondrodysplasias is comprised of the most common genetic forms of dwarfism in human. Its member, thanatophoric dysplasia types II (TDII), is caused by a distinct mutation at the kinase domain of FGFR3 which retard skeletal growth and development. However, there is no clue about the mediators of this mutant FGFR3-related growth abnormalities in development.

To study the effects of the TDII mutation on FGFR3 activity for downstream signal transduction, the expression vectors of FGFR3 with the TDII mutation (Lys650Glu) was constructed. After transient transfection into 293T cells, the FGFR3 proteins were immunoprecipitated from transfected cells, and their kinase activities were assessed by an in vitro autophosphorylation (FIG. 20A). The amount of wild type and the TDII mutant FGFR3 in the precipitate were at similar levels (FIG. 20B). However, TDII mutant FGFR3 showed a greatly elevated intrinsic kinase activity in comparison with the wild-type receptors. STAT activation in these different FGFR3 transfected cells was assayed using the electrophoresis mobility shift assay (EMSA). The expression of mutant TDII FGFR3 could induce a distinct protein complex that specifically bound to the high-affinity STAT interacting site (M67-SIE) in a labeled DNA probe (FIG. 20C). This complex co-migrated with IFN-γ induced STAT1 complex (lane 1), and was further “supershifted” by a specific anti-STAT1 antibody, but not by a control antibody, indicating that expression of this mutant TDII FGFR3 resulted in STAT1 activation. These results demonstrate that the expression of mutant TDII FGFR3 can constitutively activate STAT1. The wild-type receptor could also activate STAT1, especially when STAT1 protein was co-expressed (data not shown). However, TDII mutant receptor activated STAT1 much more strongly than wild-type FGFR3, and this pattern of different levels of STAT activation might correlate with the constitutive kinase activities of these receptors. Moreover, STAT1 activation by the TDII FGFR3 could be further enhanced by FGF 1, a ligand for FGFR3 (Ornitz and Leder, 1992) (data not shown).

The status of MAP kinase in these transfected cells was analyzed. No detectable differences in the forms of phosphorylated and unphosphorylated MAP kinases between TDII and wild-type or other mutant FGFR3 transfected cells was found, indicating that in contrast to STAT1 protein, MAP kinase might not be involved in the abnormal function of the TDII receptor.

STAT1 nuclear translocation, p21/WAF1 induction and cell growth arrest in TDII transfected cells and in chondrocytes from TDII patients. To confirm that the TDII receptor was expressed properly in the transfected cells, and to demonstrate the expression of this TDII receptor-induced STAT activation at the cellular level, intracellular localization of STAT1 in response to the expression of FGFR3 was examined. It is established that latent STAT1 protein was located in the cytoplasm, whereas activated STAT1 could translocate into the nucleus (Schindler, 1992b; Fu and Zhang, 1993). Therefore, translocation of STAT1 into the nucleus is an indication of STAT1 activation. Although not illustrated further at this point, one skilled in the art can recognize that an assay utilizing this observation could be readily developed. For example, a method of diagnosing abnormal STAT activation could involve nuclear staining of test cells using anti-STAT antibodies and the examination of the stained nuclei for evidence of STAT protein translocation into the nuclei.

As shown in cells transfected with the wild-type FGFR3, the STAT1 protein (visualized by black color) was barely recognizable, possibly due to the fact that STAT1 was expressed at a low level and normally spread out in the cytoplasm, while the FGFR3 protein was expressed on the cell surface (brown color), as indicated by double staining of the cells with an immunocytochemical method using specific anti-STAT1 and anti-FGFR3 antibodies (FIG. 21A, indicated by arrows). However, the cells transfected with the TDII construct showed the expression of TDII receptor on the membrane, and many cells showed concentrated localization of STAT1 in the nucleus (FIG. 21B, indicated by arrows), suggesting STAT1 activation in these cells. To confirm the nuclear localization of STAT1, the nuclei were counter stained by blue color with hematoxylin, while the anti-STAT1 antibody-recognized proteins were visualized by brown color, showing the overlap stainings of STAT1 and the nuclei in the TDII receptor-transfected cells (dark brown, indicated by arrows in FIG. 21C). These results further confirmed that expression of TDII mutant receptor can activate STAT, resulting in STAT1 translocation to the nucleus.

Relationship between STAT1 activation by of TDII receptor and expression p21. It was further determined whether STAT1 activation by the expression of TDII receptor would induce expression of p21. As anticipated, the p21 mRNA level was particularly enhanced in TDII-transfected cells compared with other transfected cells (FIG. 22A). Probably due to the calcium mediated transfection, p21 mRNA levels were non-specifically higher in transfected cells than in untransfected cells (Mock, FIG. 22A, lane 3). EGF induced p21 mRNA in A431 cells served as a positive control of p21 induction. Consistent with p21 mRNA expression, p21 protein was enriched in the nuclei in TDII transfected cells as demonstrated by an immunocytochemical stain with anti-p21 antibody (indicated by arrows in FIGS. 22D and 22D). Since 293T cells express adenovirus E1B and SV40 large T antigen (Pear et al., 1993), which are capable of inactivating p53, this observed p21 induction in TDII transfected cells may not involve p53 (Ko and Prives, 1996). It was further shown that the rate of DNA synthesis and cell growth were significantly reduced in TDII-transfected cells, whereas expression of the wild-type receptor did not inhibit DNA synthesis or colony formation (data not shown).

Analysis of chondrocytes in situ in growth plates from TDII affected individuals or other control individuals for possible STAT1 activation and p21 expression, were further examined using immunohistochemistry. Heterozygosity for the Lys650Glu mutation at the DNA level was previously confirmed in two TDII patients (data not shown).

As shown in FIG. 23, STAT1 was expressed at a low level in the chondrocytes from a normal control individual, and STAT1 protein was found in the cytoplasm (FIG. 23A, brown staining of STAT1 was indicated by arrows; the nuclei were counter stained in blue), indicating STAT1 is not normally activated in these cells. Strikingly, STAT1 was found to be translocated into the nuclei, and exclusively stained in the nuclei in many chondrocytes at the growth plates of femur from three TDII-affected individuals (FIG. 23B indicated by arrows), indicating STAT1 activation in these TDII chondrocytes. The arrowheads in the low magnification view on the left of each panel showed the area where the higher magnification view (on the right) was taken (indicated by arrowheads). To confirm that this nuclear stain by anti-STAT1 antibody was specifically caused by STAT1 protein, the staining procedure with or without the purified recombinant STAT1 protein as a competitor was repeated. This nuclear staining by the anti-STAT1 antibody in chondrocytes of the TDII affected patient (FIG. 23C) was completely abolished by the specific competitor (FIG. 23D), confirming specificity of STAT1 staining and the solidity of observation of nuclear localization of STAT1 in these TDII chondrocytes. This nuclear STAT1 staining pattern in the growth plate was consistent with the staining pattern of FGFR3 (data not shown), suggesting this STAT translocation was probably caused by actions of the mutant TDII receptor.

p21 expression in the same TDII-affected chondrocytes was further examined. As shown in FIG. 24, p21 protein was undetectable with an anti-p21 antibody (no brown stain) in normal chondrocytes (FIG. 24A), however, p21 expression was clearly observed in the TDII chondrocytes as indicated by brown or darker nuclear stain by the anti-p21 antibodies (FIG. 24B, indicated by arrows). p21 expression might cause growth retardation of these TDII chondrocytes, resulting in anomalies in bone development. Furthermore, as shown in FIG. 24B, the p21 staining is mostly in the nucleus (arrows). However, in some cells, the p21 staining looked to be in the cytoplasm (arrowhead). Intriguingly, there were vacuole-like structures in these cells. This vacuole-like structure indicates the cell degeneration which was frequently observed in the TDII chondrocytes. Thus it was further shown that expression of TDII mutant FGFR3 would cause cell death, especially the programmed cell death or apoptosis.

Discussion

Cytokines or growth factors, such as EGF, FGF, IFN-γ, and many interleukins, can stimulate a number of different and parallel signaling pathways. The Ras-MAP kinase and PI3 kinase signaling pathways have been implicated in mitogenic responses and cell survival. However, many cytokines have been shown to stimulate growth in one cell type, while inhibiting growth or inducing differentiation in the other cell types (Chin et al., 1996). In contrast, the STAT signaling pathway may mediate negative control of cell growth or induce apoptosis in response to extracellular stimuli as shown above. Thus, it is possible that cytokines simultaneously initiate mitogenic pathways, possibly involving the RAS protein, MAP kinases, PI3 kinase or other signaling proteins, and the STAT signaling pathway, that may negatively regulate cell growth and survival by inducing expression of cell cycle inhibitors such as p21, in addition to the apoptotic mediator ICE.

Thus whether a cytokine promotes or inhibits growth is determined by the relative strengths of the positive and negative signals that may be induced simultaneously by the same ligand (such as EGF)-bound receptor. Additionally, the relative strength of these positive or negative signals may also vary in different cell types in according to specific cell content. For example, EGF has been shown to activate the MAP kinase pathway, but not the STAT pathway in many types of cells (see above). In those cells, in the presence of the predominant positive signals to the nucleus, EGF acts as a survival or growth factor. However, in A431 and MDA-MB468 cells (Chin et al., 1997), although EGF treatment could activate the EGF receptor, MAP kinase and other downstream signaling proteins, the STAT proteins were also strongly activated by EGF in these cells, generating a negative signal, including induction of cyclin inhibitors such as p21, which overrides the positive growth signal, causing net inhibition of cell growth. Similarly, in normal development, STAT activation may be required for proper negative control to balance actions of the mitogenic pathway. Thus, cells can grow or differentiate by joint actions of both pathways. However, during abnormal pathological situations, such as TDII FGFR3, this receptor experienced abnormally higher kinase activity, causing stronger STAT1 activation, p21 induction and cell growth arrest in chondrocytes of long bone and other tissues where the FGFR3 is expressed (Peters et al., 1993). Thus, normal growth and development are disrupted.

It is well known that p53 can mediate induction of p21. However, p53 is probably not involved in p21 induction in responses to cytokines as shown here, because the level of p53 during the p21 induction by IFN-γ and EGF was not altered (data not shown) and the p53 protein in A431 cell was mutated at the codon 273 and probably nonfunctional (Kwok et al., 1994). It has recently been shown that in addition to the CDK inhibitor p27^{Kip1}, p21 is also induced in a p53-independent pathway in some types of cells treated with TGF-b (Datto et al., 1995). Therefore, the p21 gene may act as a common mediator of the negative growth signals for the genotoxic stimulation of the cell, such as radiation, as well as from actions of cytokine receptors, although the promoter elements involved in each event may be different.

One of the striking examples observed is growth inhibition mediated by the FGF receptor. Fibroblast growth factor (FGF) and its receptors (FGFRs) have crucial functions in differentiation, cell migration and development. At least nine members of FGFs have been identified. The four FGFRs also have been known to be encoded by unlinked genes (FGFR1-4) (Johnson and Williams, 1993; Mason, 1994). All these FGFRs have an extracellular region with three distinct domains which exhibit structure similarities to immunoglobulins (Ig-like), a transmembrane domain and a split cytoplasmic tyrosine kinase domain (see FIG. 1). All these domains are necessary for functions of FGFRs. Genetic mutations at these various domains could have great biological consequences (see below).

FGFs binds to these FGFRs causing activation of the intrinsic tyrosine kinase and autophosphorylation of the receptors (Bellot et al., 1991; Coughlin et al., 1988; Mohammadi et al., 1996). The tyrosine phosphorylated receptor exhibits elevated tyrosine kinase activity which can further phosphorylate intracellular signal proteins that interact with the autophosphorylated receptor (Mason, 1994). The tyrosine phosphorylation of the receptors is clearly essential for the biological functions of FGFs. Besides these high affinity FGFRs, FGFs also bind with low affinity to cell surface proteoglycans, such as heparin, which are required for generating a full biological response (Schlessinger et al., 1995).

One of the most notable functions of FGFs and FGFRs is thought to be promotion of cell growth; or in other words, FGFs can act as growth factors (Mason, 1994; Wang et al., 1994). Furthermore, some FGFs, such as FGF4, have been identified as oncogenes in a transformation assay of NIH 3T3 cells (Mason, 1994). The expression of a constitutively activated mutant FGFR3 has been shown to stimulate growth of a type of cell (Naski et al., 1996). Consistent with these observations, FGF-FGFR interaction can activate the RAS-MAP kinase pathway which may initiate mitogenic responses (MacNicol et al., 1993; Mason, 1994; Shaoul et al., 1995). PLC-g, a cellular signaling protein, also can be activated by FGF (Antonelli-Orlidge et al., 1989; Jaye et al., 1992). In addition, FGFs also can modulate cell differentiation, migration and survival (Mason, 1994). However, the mechanisms of how these downstream signal proteins can exert the variety of functions of FGFs are largely unknown. It is possible that additional signaling pathways may be required for these FGF functions.

Mutations in FGFRs have been shown to cause dominantly inherited human skeletal abnormalities (Erlebacher et al., 1995; Muenke and Schell, 1995). For example, the achondroplasia class of chondrodysplasias is comprised of the most common genetic forms of dwarfism in human. Its members, achondroplasia (ACH), hypochondroplasia (HCH) and thanatophoric dysplasia types I and II (TDI and TDII), are caused by distinct mutations of fibroblast growth factor receptor 3 (FGFR3) which retard skeletal growth and development (FIG. 1, see below). However, there is no reported connection about the mediators of these mutant FGFR3-related growth abnormalities in development.

TDII is a lethal form of dwarfism that results from a recurrent point mutation (Lys650Glu) within the activation loop of tyrosine kinase domain (Tavormina et al., 1995). Its phenotype is characterized by severe shortening of limbs, thin vertebral bodies, and skull anomalies and its histopathology by disrupted proliferation and organization of the cartilaginous growth plates of long bones (Gorlin et al., 1990; Sillence et al., 1979). ACH is associated with a Gly380Arg substitution in the transmembrane domain of FGFR3 (Rousseau et al., 1994; Shiang et al., 1994), and HCH is related to a recurrent mutation in a distinct region of tyrosine kinase domain (Bellus et al., 1995). ACH and HCH are not fatal diseases; they represent similar, but milder phenotypes than those of TDII in the retardation of bone growth.

Mutant FGFR3, such as ACH, HCH, and TDII, can negatively regulate chondrocyte proliferation. More importantly, the negative effects are not caused by loss of the signaling function of FGFR3. On the contrary, all these FGFR3 mutations are gain-of-function mutations which produce a dominant activating effect, especially the constitutively activated tyrosine kinase activities (Muenke and Schell, 1995; Naski et al., 1996; Webster and Donoghue, 1996). In support of the notion of negative regulation of cell proliferation by FGFR3, homozygous disruption of FGFR3 in mice causes severe and progressive bone dysplasia with enhanced growth, further indicating that FGFR3 is a negative regulator of bone growth (Colvin et al., 1996; Deng et al., 1996). On the basis of these in vivo data, the logical explanation for phenotypes of the chondrodysplasias is that the negative regulation of cell growth by FGFR3 is abnormally elevated in the chondrocytes that express the gain-of-function mutant FGFR3, such as ACH, HCH, TDI and TDII, causing retardation in bone growth.

Thus, one of the biological effects of these FGFR3 mutants at the cellular level is inhibition or retardation of chondrocyte proliferation in the cartilaginous growth plates. To understand the molecular pathogenesis of these genetic diseases, the identify of the mediators of this negative control through mutant FGFR3 signal transduction during bone growth must be determined. In Example 2, the mediator of the activity of mutant FGFR3 was shown to be activated STAT protein.

Furthermore, as shown in FIG. 25, a number of other important tyrosine kinases are well conserved in the activation loop of the kinase domain. The present inventors predict that mutations at the conserved Lys or Arg residues (boxed) results in constitutive activation of the kinases. One possible substrate(s) may be STAT1 or other STAT proteins. This mode of activation and the downstream signaling are involved in the pathogenesis of several mammalian diseases, and can therefore serve as a target for both diagnosing the disorder as well as a therapeutic target for agent development.

The present inventors found that there is no significant difference in the state of phosphorylation of MAP kinases among the 293T cells transfected with the TDII or other vectors (unpublished result). Intriguingly, the initial studies of STAT1 null mice showed no overt defect in the development (Meraz et al. 1996; Durbin et al. (1996). This might be due to the redundancy in the STAT family proteins or to the possibility that STAT1 can be activated only when the FGFR3 has the constitutively activating mutation. In this context, it is noteworthy that either the ACH or HCH receptor showed a weak activation of STAT1 in the transfection assay, which may correlate with milder phenotypes of ACH and HCH than TDII (unpublished result). This is the first example of human genetic disease which involves an abnormality in the tyrosine kinase-STAT pathway. This observation can therefor form a basis of developing diagnostic and therapeutic agents for use in TDII and other FGFR associated disease (see Muenke et al. 1995).

On the basis of the observations provided in these Examples, the present inventors suggest that their recently discovered STAT signaling pathway may be involved in negative regulation of cell growth (Chin et al., 1996). This invention provides methods for developing agents that can be used to diagnosis or treat growth abnormalities, such as TDII, that are regulated by the way of STAT activation.

### EXAMPLE 3

In parallel to the known mitogenic pathways mediated by signal proteins such as Grb2, many cytokines can also activate STAT proteins which mediate direct signal transduction and gene expression. However, it is not known whether STAT proteins can be activated by FAK.

The data presented herein indicate that FAK may activate in vivo a negative signaling pathway involving STAT. Strikingly, it was found that in contrast to many other signaling pathways initiated by FAK, the FAK-STAT signaling pathway may negatively regulate cell attachment.

STAT1 can interact with FAK in the transfected cells STAT proteins can bind directly to phosphorylated receptor-tyrosine kinase complexes (Greenlund et al. (1994)). To determine how FAK can activate STAT1, the present inventors examined interactions between STAT1 and FAK. STAT1 was immunoprecipitated by an anti-STAT1 antibody from lysates of 293T cells co-transfected with STAT1 and FAK. The co-immunoprecipitated proteins were analyzed by a Western blot with an anti-HA-tag antibody that could recognize both exogenously expressed STAT and FAK (both proteins were HA-tagged) (FIG. 26). The FAK protein was co-immunoprecipitated with the anti-STAT1 antibody. The identity of the HA-tagged FAK was confirmed further by blotting with an anti-FAK antibody. The expression levels of STAT1 protein were also assayed (FIG. 26, lower panel). These results suggest that STAT1 and FAK are associated with each other in these STAT1 and FAK co-transfected cells.

STAT:FAK interactions in untransfected cells were examined. Complementary to the above co-immunoprecipitation with an anti-STAT1 antibody described above, an anti-FAK antibody was used to perform immunoprecipitation in untransfected 293T cells. Then the immunoprecipitated complexes were further examined using an anti-STAT1 antibody in a Western blot. In this assay, STAT1 was clearly co-immunoprecipitated with anti-FAK antibody. The co-immunoprecipitated STAT1 migrated slightly slower than the major STAT1 band (indicated as STAT1, FIG. 27A, lane 1-4), immunoprecipitated by the anti-STAT1 antibody (FIG. 27A, lane 5). The more slowly migrating STAT1 bands resulted from protein phosphorylation after they had interacted with FAK. This notion was confirmed by Western blot with an antibody that specifically recognizes tyrosine phosphorylated, but not unphosphorylated, STAT1 (New England Biolab). Only these slower migrating bands were recognized by this anti-phospho-tyrosine STAT1 (STAT1p) antibody (FIG. 27A, lanes 6-10), while the major unphosphorylated STAT1 band shown in lane 6, was not recognized by this antibody (FIG. 27A, lane 10). Intriguingly, it appeared that only tyrosine-phosphorylated STAT1 was co-immunoprecipitated with FAK, and this FAK-STAT association transiently reached the maximal level when cells were just attached to fibronectin (at 0.5 hour time point, lanes 2 and 7), a ligand for the integrin receptor which could activate FAK during cell adhesion. With the progression of cell attachment, the amount of STAT1 associated with FAK was significantly reduced. These results suggest that STAT1 can associate transiently with FAK at the beginning of cell adhesion when FAK is activated.

A similar observation was also made in A431 cells. Consistent with the transient STAT1 association with FAK and STAT1 tyrosine phosphorylation, a specific STAT1 DNA binding activity was observed at the beginning of the cell attachment and this STAT1 activity was reduced gradually as the cell attachment proceeded (FIG. 27B). These data suggest that STAT1 can be transiently activated during cell adhesion.

The above observations provides a target for developing agents that stimulate or block cell adhesion by interfering with STAT-FAK interaction (see below).

The specificity of activation of STAT1 by FAK was confirmed by using various STAT1 and FAK mutants. Mutations of the SH2 domain (STAT1-SH2RQ) and of the tyrosine 701 (STAT1-CYF) in STAT1 prevented its activation when co-transfected with FAK (FIG. 28). Almost equal STAT1 protein levels in various transfected cells were verified by Western blotting (FIG. 28, lower panel). Similarly, expression of wild-type STAT1 in these cells occasionally resulted in a low level of STAT1 activation; however, expression of SH2 mutants (STAT1-SH2RQ, and -CYF) alone did not generate this STAT1 activation. These results indicate that the SH2 domain and tyrosine 701 are essential for STAT1 activation by FAK.

STAT1 and FAK co-expression causes cell detachment. The present inventors were further interested in the possible cellular effects of STAT1 activation by FAK. Intriguingly, dramatic morphological changes in transfected cells that seemed to increase with the increased level of STAT1 activation by FAK were observed (FIG. 29). In contrast to mock transfected cells, many cells which had been transfected with FAK and STAT were detached from the plate or aggregated. In cells transfected with STAT1 alone, there was little effect on cell morphology, whereas in cells transfected with FAK alone, there was a certain degree of similar morphological alterations, which may correlate with the endogenous STAT1 activity induced by FAK. These results suggest that the joint action by FAK and STAT1 have significant effects on cell morphology.

One possible mechanism for these morphological changes is that STAT1 activation by FAK could negatively affect cell adhesion, thus providing a target for developing agents that modulate cell adhesion. Although FAK has been suggested to be involved in focal adhesion in normal conditions (Richardson et al. 1996), STAT activation may trigger another signaling pathway causing negative effects on cell adhesion, similar to the negative effects on cell growth induced by EGF through STAT activation.

To test this possibility, the present inventors determined the ability of these transfected cells to adhere to fibronectin, the ligand for integrin that can activate FAK (FIG. 29). To accomplish this, Fibronectin (1 ug) was adsorbed onto plastic 96-well tissue culture plates. After using 0.5% BSA to block the plates in 37° C., certain numbers of cells were plated and incubated at 37° C. for 3 hours. Plates were washed with PBS twice and cells fixed with 3% paraformaldehyde (pH 7.4) for 30 min. at 4 C. Cells were stained with 0.5% crystal violet and incubated overnight at room temp. The relative extent of cell adherence was determined by OD630.

Co-expression of FAK and STAT1 in 293T cells greatly inhibited the cell adhesion on fibronectin. Expression of either STAT1 or mock expression of b-galactosidase, or STAT1-SH2RQ mutant, had less effect. Thus, although FAK alone has certain negative effects on cell attachment, both STAT and FAK are required for the maximal inhibition of cell adhesion in this cell transfection system. While not explained further at this point, one of ordinary skill in the art can readily envision assay methods based on agents which either inhibit or promote STAT and FAK activity and expression, either separately or together. These assay methods can be used to identify agents which either increase or decrease the inhibition of cell adhesion by altering STAT and FAK levels and activities.

STAT1 is required for cell detachment. Endogenous FAK-STAT can be activated by cell attachment to FN. To confirm that the STAT protein is involved in detachment of cells, U3A cells which are defective in STAT1 protein, and three U3A derived cell clones that expressed reintroduced STAT1 protein (U3A-Stat1, clones 2.1, 2.3, 2.9) were analyzed for cell adherence on fibronectin (FN). The re-introduction of STAT1 protein to U3A cells significantly reduced cell attachment to fibronectin (FIG. 30A). The results presented were an average of three repeated experiments.

Embryonic fibroblasts derived from STAT1 deficient mice or from wild type mice from the same litter also were compared. STAT1 null (−/−) cells attached better than STAT1 +/+ cells at different concentrations of plated fibronectin (FIG. 30B).

FIG. 30C shows that STAT1 wild-type fibroblast cells were detached and aggregated on a plate coasted with FN, whereas STAT1−/− cells could attach well at the same conditions.

STAT1 promotes cell migration. STAT1−/− and STAT1 +/+ fibroblasts were further analyzed using a Boyden chamber assay for their migration ability. It was found that STAT1 positive cells migrate significantly faster than STAT1 negative cells, indicating that STAT1 can promote cell migration (FIG. 31).

In summary, cell adhesion and anchorage are necessary for growth and survival of most types of cells (Fang et al. (1996); Meredith et al. (1993); Frisch et al. (1994); and Rouslahts et al. (1994)). However, the present invention demonstrates the surprising results that the STAT signaling pathway can also be activated by FAK, resulting in a negative effect on cell attachment and enhancement of cell migration. The above observations may be relevant to the pathogenesis of many diseases that are caused by abnormal cell detachment and migration. The above experiments in this invention provide tools and methods for developing assay systems and screening for agents that modulate STAT:FAK activation and subsequently, cell attachment and migration.

### EXAMPLE 4

Methods to identify agents that block or stimulate the phosphorylation of RECEPTOR/PTK-STAT may utilize any commonly available tyrosine kinase assays to assay for the modulation of phosphorylation. Such assays are widely available such as those disclosed by Ruksen et al., “Nonradioactive Assays of Protein-Tyrosine Kinase Activity Using Anti-phosphotyrosine Antibodies” Methods in Enzymology 200:98-107 (1991) or Sahal et al., “Solid-Phase Protein-Tyrosine Kinase Assay” Methods in Enzymology 200:90-97 (1991),

For instance, a STAT protein or fragment thereof is incubated with a tyrosine kinase such as FAK, Itk, TIE or Src tyrosine kinases and ³²P-labeled gamma-ATP in the presence and absence of the agent to be tested. The samples are then contacted with anti-STAT antibodies immobilized on a column; the column is washed; and the bound STAT protein or fragment is eluted with 0.1M glycine, pH 2.5. The eluant is then subjected to fractionation to separate the resulting radiolabeled STAT protein or fragment from the free radioactivity in the sample using any conventional technique, such as precipitation in 5-10% trichloroacetic acid. Following fractionation, the amount of radioactivity incorporated into the STAT protein or fragment is counted. Comparing the amount of radioactivity incorporated into the STAT protein or fragment in the presence and absence of the agent to be tested identifies an agent which modulates, blocks or stimulates the phosphorylation of RECEPTOR/PTK-STAT. Inhibition of STAT phosphorylation indicates the potential to inhibit apoptosis while the promotion of STAT phosphorylation indicates the potential to stimulate or promote apoptosis.

In an alternative assay format, 100 ng of a STAT protein or fragment is added to 1 ml buffered solution containing a tyrosine kinase such as a FAK, Itk, TIE or Src tyrosine kinases, together with 30 μC ³²P-gamma ATP in the presence or absence of the agent to be tested. Following incubation, the mixture is heated to 100° C. in a solution containing sodium lauryl sulfate (SDS) and beta-mercaptoethanol. Aliquots are electrophoresed on 10-15% gradient SDS polyacrylamide gels and exposed to X-Omat X-ray film to identify radioactive STAT protein or fragments. Comparing the amount of radioactivity incorporated into the STAT protein or fragment in the presence and absence of the agent to be tested identifies an agent which modulates, blocks or stimulates the phosphorylation of RECEPTOR/PTK-STAT. Inhibition of STAT phosphorylation indicates the potential to inhibit apoptosis while the promotion of STAT phosphorylation indicates the potential to stimulate or promote apoptosis.

### EXAMPLE 5

Uses for Agents which Modulate STAT-Mediated Apoptosis.

As discussed herein, RECEPTOR-PTK-STAT pathways play an important role in a wide variety of intracellular events, disease processes, cell morphology, and intracellular interactions including cellular attachment, cellular aggregation and cellular migration. In addition, these RECEPTOR-PTK-STAT pathways play an important role in the apoptosis process. Agents that modulate, reduce or block the interactions of a receptor with its associated phosphotyrosine kinases or block the sequential interactions with members of the STAT family of proteins can be used to modulate biological and pathologic processes associated with the RECEPTOR-PTK-STAT pathways.

The data presented herein demonstrates the involvement of tyrosine kinases with a variety of receptors that participate in modulating the STAT-mediated apoptotic process. Agents that modulate, e.g., cell mortality, cell migration and/or intercellular interactions may therefore, also control pathologic cell growth migration such as tumor metastasis or chronic inflammation.

As used herein, a subject can be any mammal, so long as the mammal is in need of modulation of a pathological or biological process effected by the cascade of intracellular events that follow the various RECEPTOR-PTK-STAT pathways. The term “mammal” is meant an individual belonging to the class Mammalia. The invention is particularly useful in the treatment of human subjects.

As used herein, a biological or pathological process mediated by STAT proteins and involving the various RECEPTOR-PTK-STAT pathways refers to the wide variety of cellular events in which a STAT protein modulates the apoptosis process or other observable or detectable intracellular events, including morphological changes, and various other biological processes. These latter processes include, but are not limited to, cellular attachment or adhesion to substrates and other cells, cellular aggregation, cellular migration, cell proliferation, and cell differentiation.

As used herein, the phrases “pathological state” or “pathological condition” in reference to events that are mediated by STAT proteins and involving the various RECEPTOR-PTK-STAT pathways includes, but is not limited to Thanatophoric Dysplasia Type II, cancer, metastasis of cancer cells, autoimmune disorders, diabetes, degenerative diseases, aging, and inflammation. A variety of cardiac and circulatory diseases also may involve such STAT proteins and the various RECEPTOR-PTK-STAT pathways including thrombosis, inflammation, angiogenesis, wound healing (including cutaneous wounds such as burn wounds, donor site wounds from skin transplants and cutaneous, decubitis, venous stasis and diabetic ulcers), acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolus, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent or cardiac valve, and conditions requiring the fitting of prosthetic devices.

Pathological processes refer to a category of biological processes which produce a deleterious effect. For example, thrombosis is the deleterious attachment and aggregation of platelets while metastasis is the deleterious migration and proliferation of tumor cells. These pathological processes can be modulated using agents which reduce or block the intracellular effects of STAT proteins and/or the various RECEPTOR-PTK-STAT pathways.

As used herein, an agent is said to modulate a pathological process when the agent reduces the degree or severity of the process. For example, an agent is said to modulate thrombosis when the agent reduces the attachment or aggregation of platelets.

Methods of Treating Pathological Conditions.

The agents of the present invention can be provided alone, or in combination with other agents that modulate a particular pathological process. For example, an agent of the present invention that modulates or increases apoptosis of an abnormal cell by increasing the amount of phosphorylated RECEPTOR/PTK-STAT proteins or by facilitating their translocation into the cell nucleus can be administered in combination with other therapeutic agents. As used herein, two agents are said to be administered in combination when the two agents are administered simultaneously or are administered independently in a fashion such that the agents will act at the same time.

The agents of the present invention can be administered via parenteral, subcutaneous, intravenous, intramuscular, intraperitoneal, transdermal, or buccal routes. Alternatively, or concurrently, administration may be by the oral route. The dosage administered will be dependent upon the age, health, and weight of the recipient, kind of concurrent treatment, if any, frequency of treatment, and the nature of the effect desired.

The present invention further provides compositions containing one or more agents which either increase or decrease the rate or extent of apoptosis in a treated cell, tissue or subject. While individual needs vary, determination of optimal ranges of effective amounts of each component is within the skill of the art. Typical dosages comprise about 0.1 to 100 μg/kg body wt. The preferred dosages comprise about 0.1 to 10 μg/kg body wt. The most preferred dosages comprise about 0.1 to 1 μg/kg body wt. However, these dosage ranges will vary according to the chemical class and overall activity of the agents to be administered, as will be appreciated by one skilled in the art.

In addition to the pharmacologically active agent, the compositions of the present invention may contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient.

Suitable formulations for oral administration include hard or soft gelatin capsules, pills, tablets, including coated tablets, elixirs, suspensions, syrups or inhalations and controlled release forms thereof.

In practicing the methods of this invention, the compounds of this invention may be used alone or in combination, or in combination with other therapeutic or diagnostic agents. In certain preferred embodiments, the compounds of this invention may be coadministered along with other compounds typically prescribed for these conditions according to generally accepted medical practice, such as anticancer agents. The compounds of this invention can be utilized in vivo, ordinarily in mammals, such as humans, sheep, horses, cattle, pigs, dogs, cats, rats and mice, or in vitro.

It should be understood that the foregoing discussion and examples present merely present a detailed description of certain preferred embodiments. It therefore should be apparent to those of ordinary skill in the art that various modifications and equivalents can be made without departing from the spirit and scope of the invention. All articles, patents and patent applications that are identified above are incorporated by reference in their entirety.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### REFERENCES

Each of the following references and all other references cited above are herein incorporated by reference:

Alnemri, E. S. et al. (1996) Cell 87, 171.

Armstrong, D. K. et al. (1994) Cancer Res 54, 5280-3.

Beg A. A., et al. Science 274, 782-784 (1996).

Bennett, M. R. et al. (1994) Circ Res 74, 525-36.

Boldin, M. P. et al. (1996) Cell 85, 803-815.

Brabyn, C. J. et al. (1995) Cell Signal 7, 139-50.

Buick, R. N. et al. (1991) Cancer Treatment & Research 53, 159-170.

Calalb M. B., et al. Mol. Cell. Biol. 15, 954-63 (1995).

Cantley, L. C. et al. (1991) Cell 64, 281-300.

Cao X. M., et al. Mol. Cell. Biol. 16, 1595-1603 (1996).

Casano, F. J. et al. (1994) Genomics 20, 474-81.

Cerretti, D. P. et al. (1994) Genomics 20, 468-73.

Chin, Y. E. et al. (1996) Science 272, 719-722.

Chin Y. E. et al., (1997) Mol. Cell. Biol. 17, 5328-5337.

Clarke, A. R. et al. (1993) Nature 362, 849-852.

Cleveland, J. L. et al. (1995) Cell 81, 479-482.

Colvin, J. S., et al. Nature Genet. 12, 390-397 (1996).

Darnell, J. E., Jr. et al. (1994) et al. Science 264, 1415-1421.

David, M., et al. (1995) Science 269, 1721-3.

DeMaeyer, E. et al. (1988) Interferons and other regulatory cytokines (New York: John Wiley and Sons).

Deng, C., et al. Cell 84, 911-921 (1996).

Dolle, R. E. et al. (1994) J Med Chem 37, 563-4.

Durbin, J. E., et al. Cell 84, 443-450 (1996).

Ellis, R. E. et al. (1991) Annu. Rev. Cell Biol. 7, 663-698.

Enari, M., et al. (1996) Nature 380, 723-6.

Englert, C., et al. (1995) Embo J 14, 4662-75.

Fang F., et al. Science 271, 499-502 (1996).

Flati, V., et al. (1996) EMBO J. 15, 1566-1571.

Fraser, A., et al. (1996) Cell 85, 781-784.

Frisch S. M., et al. J Cell Biol 124, 619-26 (1994).

Fu, X.-Y. et al. Cell 74, 1135-1145 (1993).

Fu, X.-Y. Cell 70, 323-335 (1992).

Fu, X.-Y. (1995a) In Inducible Gene Expression, P. Bauerle, ed. (Boston: Birkhauser), pp. 99-130.

Fu X. Y., J. Leukoc. Biol. 57, 529-35 (1995).

Gill, G. N., et al. (1981) Nature 293, 305-307.

Greenlund A. C., et al. EMBO J. 13, 1591-600 (1994).

Guan J. L., et al. Nature 358, 690-2 (1992).

Hanks S. K., et al. Proc. Natl. Acad. Sci. 89, 8487-91 (1992).

Harrington, E. A., et al. (1994) Embo J 13, 3286-95.

Hildebrand J. D., et al. J Cell Biol 123, 993-1005 (1993).

Hsu, H., et al. (1995) Cell 81, 495-504.

Hynes R. O., Cell 69, 11-25 (1992).

Ihle J. N., et al. Rev. Immunol. 13, 417-435 (1995).

Ihle, J. N. et al. Trends Genet 11, 69-74 (1995).

Johnson, M. D., et al. (1993) Cancer Research 53, 873-877.

Jung, Y., et al. (1996) J Biol Chem 271, 5112-7.

Juo, P., et al. (1997) et al. Mol. Cell. Biol. 17, 24-35.

K. Shuai, et al. Science 261, 1744-6 (1993).

Karin, M., et al. (1995) et al. Curr. Biol. 5, 747-757.

Kawamoto, T., et al. (1984) J Biol Chem 259, 7761-7766.

Kimchi, A. (1992) J. Cell. Biochem. 50, 1-9.

Ko, L. O. et al. Genes Dev. 10, 1054-1072 (1996).

Kuida, K., et al. (1995) Science 267, 2000-3.

Kumar, A., et al., Science 278, 1630-1632 (1997).

Larner A. C., et al., Science 261, 1730-1733 (1993).

Larner, A. C. et al. Science 261, 1730-1733 (1993).

Leaman D. W., et al., Mol Cell Biol 16, 369-75 (1996).

Leonard, W. J. et al., Annu. Rev. Immunol. 16, 293-322 (1998).

Levy, D. E., et al. (1989) et al. Genes Dev. 3, 1362-1371.

Li, P., et al. (1995) et al. Cell 80, 401-411.

Loh J. E., et al., Mol. Cell. Biol. 0.14, 2170-9 (1994).

Lowe, S. W., et al. (1993) et al. Nature 362, 847-849.

MÅller M., et al., Nature 366, 129-135. (1993).

Marshall, C. J. Cell 80, 179-185 (1995).

Martin, S. J., et al. (1995) et al. Cell 82, 349-352.

Mason, I. J. Cell 78, 547-552 (1994).

McKendry, R., et. al. (1991) et al. Proc. Nat. Acad. Sci. USA. 88, 11455-9.

Meraz, M. A. et al. Cell 84, 431-442 (1996).

Meredith J. E., Jr., et al. Mol Biol Cell 4, 953-61 (1993).

Miura, M., et al. (1993) et al. Cell 75, 653-60.

Mizushima, S. et al. Nucleic Acids Res. 18, 5332 (1990).

Muenke, M. et al. Trends Genet. 11, 308-313 (1995).

Muzio, M., et al. (1996) et al. Cell 85, 817-827.

Nagata, S., et al. (1995) et al. Science 267, 1449-1456.

Naski, M. C., et al. Nature Genet. 13, 233-237 (1996).

Nicoletti I., et al. J. Immunol. Methods 139, 271-9 (1991).

Oberhammer, F. A., et al. (1992) et al. Proc Natl Acad Sci U S A 89, 5408-12.

Ornitz, D. M. et al. J. Biol. Chem. 267, 16305-16311 (1992).

Pawson, T. (1994) Adv Cancer Res 64:87-110.

Pear W. S., et al. Proc. Natl. Acad. Sci. USA 90, 8392-8396. (1993).

Peters, K., et al. Dev. Biol. 155, 423430 (1993).

Pine, R., et al. (1994) et al. Embo J 13, 158-67.

Quelle F. W., et al., J. Biol Chem. 270, 20775-80 (1995).

Raff, M. C. (1992) Science 356, 397-400.

Richardson A., et al. Nature 380, 538-40 (1996).

Ruff-Jamison S., et al. Proc. Natl. Acad. Sci. USA 92, 4215-4218 (1995).

Ruff-Jamison, S., et al. (1993) Science 261, 1733-1736.

Ruoslahti E., et al. Cell 77, 477-8 (1994).

Sadowski, H. B. et al. Nature 362, 79-83 (1993).

Sadowski, H. B., et al. (1993) et al. Science 261, 1739-1744.

Schaller M. D., et al. Proc. Natl. Acad. Sci. 89, 5192-6 (1992).

Schaller M. D., et al. J. Cell. Biol. 130, 1181-7 (1995).

Schaller M. D., et al., Mol Cell Biol 14, 1680-8 (1994).

Schindler, C., et al. Science 257, 809-813 (1992).

Schlaepfer D. D., et al. Nature 372, 786-91 (1994)

Schlessinger, J. et al. Neuron 9, 383-391 (1992).

Schwartz M. A., et al. Annu. Rev. Cell. Dev. Biol. 11, 549-599 (1995).

Shi, L., et al. (1996) Proc. Natl. Acad. Sci. USA 93, 11002-11007.

Shuai, K., et al. Science 259, 1808-1812 (1992).

Shuai, K., et al. (1993) et al. Nature 366, 580-3.

Sillence, D. O., et al. Am. J. Pathol. 96, 813 (1979).

Sporn, M. B., et al. (1988) Nature 332, 217-219.

Stanger, B. Z., et al. (1995) Cell 81, 513-523.

Steller, H. (1995) Science 267, 1445-1449.

Su et al. (1997) Nature 386, 288-292.

Tamura, T., et al. (1995) Nature 376, 596-9.

Tartaglia, L. A., et al. (1993) et al. Cell 74, 845-853.

Tavormina, P. L. et al. Nature Genet. 9, 321-328 (1995).

Tewari, M., et al. (1995) Cell 81, 801-9.

Thompson, C. B. (1995) Science 267, 1456-1462.

Tsujii M., et al. Cell 83, 493-501 (1995)

van der Geer, P., et al. Annu. Rev. Cell. Biol. 10, 251-337 (1994).

Van Antwerp D. J., et al. Science 274, 787-789 (1996)

Velazquez, L., et al. Cell 70, 313-322 (1992).

Wang C.-Y., et al. Science 274, 784-787 (1996)

Webster, K. M., et al. Mol. Cell. Biol. 16, 4081-4087 (1996).

Webster, M. K. et al. EMBO J. 15, 520-527 (1996)

White, E. (1996) Genes Dev 10, 1-15.

Wyllie, A. H., et al. (1980) Int Rev Cytol 68, 251-270.

Xia, K., et al. (1996) Proc Natl Acad Sci 93, 11681-11686.

Xie B., et al., unpublished data

Xu, X., et al. (1996) et al. Science 273, 794-797.

Yamamoto, H., et al. J. Exp. Cell Res. 222, 125-130 (1996)

Yu C. L., et al., Science 269, 81-3 (1995)

Yuan, J., et al. (1993) et al. Cell 75, 641-52.

Z. Liu, et al. Cell 87, 565-575 (1996)

Zhong, Z., et al. (1994) Science 264, 95-8.

What is claimed:

1. A method of modulating the rate and/or amount of a cellular process selected from the group consisting of cell growth, cell detachment and cell migration, and cellular apoptosis, said method comprising altering the RECEPTOR/PTK-STAT pathway of a cell.

2. The method of claim 1 wherein the RECEPTOR/PTK-STAT pathway is altered by increasing or decreasing the amount of phosphorylated RECEPTOR/PTK-STAT proteins present in a cell.

3. The method of claim 2 wherein the amount of phosphorylated RECEPTOR/PTK-STAT proteins present in the cell is increased by introducing into the cell a nucleic acid molecule that encodes a tyrosine kinase.

4. The method of claim 2 wherein the amount of phosphorylated RECEPTOR/PTK-STAT proteins present in the cell is increased by introducing into the cell a nucleic acid molecules that encodes RECEPTOR/PTK-STAT proteins.

5. The method of claim 1 wherein the RECEPTOR/PTK-STAT pathway is altered by increasing or decreasing the expression and/or activation of a RECEPTOR in the pathway.

6. The method of claim 1 wherein the RECEPTOR/PTK-STAT pathway is altered by increasing or decreasing the amount of STAT in a cell.

7. The method of claim 6 wherein the STAT is selected from the group consisting of STAT1, STAT3, STAT4, STAT5A/B and STAT6.

8. The method of claim 1 wherein the RECEPTOR/PTK-STAT pathway is altered by altering the interaction of STAT with a RECEPTOR in the pathway.

9. The method of claim 1 wherein the RECEPTOR/PTK-STAT pathway is altered by altering the interaction of STAT with a PTK.

10. The method of claim 9 wherein the PTK is selected from the group consisting of a cellular kinase, a receptor tyrosine kinase, and a cytoplasmic tyrosine kinase.

11. The method of claim 9 wherein the PTK is selected from the group consisting of EGFR, FGFR, FAK, JAK, Src, Lck, TIE2, c-kit, RET, INRK, EPH, TRKA, TRKB, Itk and PDGFR-B.

12. The method of claim 11 wherein the EGFR is selected from the group consisting of HER1, HER2, HER3 and HER4.

13. The method of claim 1 wherein the RECEPTOR/PTK-STAT pathway is altered by increasing or decreasing the amount of kinase present in a cell.

14. The method of claim 13 wherein the kinase is selected from the group consisting of a cellular kinase, a receptor tyrosine kinase, or a cytoplasmic tyrosine kinase.

15. The method of claim 13 wherein the kinase is selected from the group consisting of EGFR, FGFR, FAK, JAK, Src, Lck, TIE2, c-kit, RET, INRK, EPH, TRKA, TRKB, Itk and PDGFR-B.

16. The method of claim 15 wherein the EGFR is selected from the group consisting of HER1, HER2, HER3 and HER4.

17. The method of claim 1 wherein the rate and/or amount of cell growth is decreased.

18. The method of claim 17 wherein the RECEPTOR/PTK-STAT pathway is altered by increasing the amount of phosphorylated RECEPTOR/PTK-STAT proteins present in a cell.

19. The method of claim 1 wherein the rate and/or amount of cell growth is increased.

20. The method of claim 19 wherein the RECEPTOR/PTK-STAT pathway is altered by decreasing the amount of phosphorylated RECEPTOR/PTK-STAT proteins present in a cell.

21. The method of claim 1 wherein the rate and/or amount of cell detachment and cell migration is decreased.

22. The method of claim 21 wherein the RECEPTOR/PTK-STAT pathway is altered by decreasing the amount of phosphorylated RECEPTOR/PTK-STAT proteins present in a cell.

23. The method of claim 1 wherein the rate and/or amount of cell detachment and cell migration is increased.

24. The method of claim 23 wherein the RECEPTOR/PTK-STAT pathway is altered by increasing the amount of phosphorylated RECEPTOR/PTK-STAT proteins present in a cell.

25. The method of claim 1 wherein the rate and/or amount of cellular apoptosis is decreased.

26. The method of claim 25 wherein the RECEPTOR/PTK-STAT pathway is altered by decreasing the amount of phosphorylated RECEPTOR/PTK-STAT proteins present in a cell.

27. The method of claim 1 wherein the rate and/or amount of cellular apoptosis is increased.

28. The method of claim 27 wherein the RECEPTOR/PTK-STAT path way is altered by increasing the amount of phosphorylated RECEPTOR/PTK-STAT proteins present in a cell.

29. A method of identifying agents which inhibit apoptosis in a cell through the mechanism of blocking the phosphorylation of RECEPTOR/PTK-STAT by a tyrosine kinase comprising the steps of:
a) incubating STAT, or a fragment thereof, and a tyrosine kinase, or a fragment thereof, with an agent to be tested,;
b) determining whether said agent blocks the phosphorylation of STAT, or a fragment thereof by said tyrosine kinase,
wherein the inhibition of STAT phosphorylation indicates the potential to inhibit apoptosis.

30. A method of identifying agents which stimulate or promote apoptosis in a cell through the mechanism of stimulating the phosphorylation of RECEPTOR/PTK-STAT by a tyrosine kinase comprising the steps of:
a) incubating STAT, or a fragment thereof, and a tyrosine kinase, or a fragment thereof, with an agent to be tested, and
b) determining whether said agent stimulates the phosphorylation of STAT, or a fragment thereof by said tyrosine kinase,
wherein the promoting of STAT phosphorylation indicates the potential to stimulate or promote apoptosis.

31. A method to assay for STAT-mediated apoptosis comprising the steps of determining whether a RECEPTOR/PTK-STAT protein is phosphorylated and correlating said apoptosis with the presence and degree of said RECEPTOR/PTK-STAT phosphorylation, wherein an increase of RECEPTOR/PTK-STAT phosphorylation indicates STAT-mediated apoptosis.

32. The method of claim 31 wherein the presence of elevated levels of RECEPTOR/PTK-STAT proteins is a diagnostic marker of Thanatophoric Dysplasia Type II, FGF-receptor associated diseases, cancer, metastasis of cancer cells, autoimmune disorders, diabetes, degenerative diseases, aging, and inflammation.

33. The method of claim 31 further comprising the steps of:
a) preparing an extract of a cell,
b) examining the proteins of said cell extract to determine the presence of a phosphorylated RECEPTOR/PTK-STAT protein, and
c) examining cellular localization of STAT protein to determine activation of STATs.

34. The method of claim 31 further comprising the steps of:
a) preparing an extract of a cell
b) examining the mRNA of said cell extract to determine the presence of a FGFR3 encoding mRNA, and
c) examining cellular localization of STAT protein to determine activation of STATs.

35. A method of treating mammalian diseases or developmental defects caused by abnormal cell death induction wherein the method comprises promoting apoptosis by altering the RECEPTOR/PTK-STAT pathway.

36. The method of claim 35 wherein the RECEPTOR/PTK-STAT pathway is altered by increasing the amount of phosphorylated RECEPTOR/PTK-STAT proteins present in a cell.

37. The method of claim 36 wherein the treatment comprises administering an agent that increases the amount of phosphorylated RECEPTOR/PTK-STAT proteins present in a cell.

38. The method of claim 36 wherein the treatment consists of a gene-therapeutic method that increases the amount of phosphorylated RECEPTOR/PTK-STAT proteins present in a cell.

39. The method of claim 35 wherein the mammalian disease or developmental defect is selected from the group consisting of cancer, autoimmune disease, viral susceptibility, and conditions of obesity.

40. A method of treating mammalian diseases or developmental defects caused by abnormal cell death induction wherein the method comprises inhibiting apoptosis by altering the RECEPTOR/PTK-STAT pathway.

41. The method of claim 40 wherein the RECEPTOR/PTK-STAT pathway is altered by decreasing the amount of phosphorylated RECEPTOR/PTK-STAT proteins present in a cell.

42. The method of claim 41 wherein the treatment consists of administering an agent that decreases the amount of phosphorylated RECEPTOR/PTK-STAT proteins present in a cell.

43. The method of claim 41 wherein the treatment consists of a gene-therapeutic method that decreases the amount of phosphorylated RECEPTOR/PTK-STAT proteins present in a cell.

44. The method of claim 40 wherein the mammalian disease or developmental defect is selected from the group consisting of degenerative disorders, ischemic injuries, viral infection induced by cell death and cell apoptosis during inflammatory responses.

45. A method of treating mammalian diseases or developmental defects caused by abnormal cell proliferation wherein the method comprises inhibiting abnormal cell growth by altering the RECEPTOR/PTK-STAT pathway.

46. The method of claim 45 wherein the RECEPTOR/PTK-STAT pathway is altered by increasing the amount of phosphorylated RECEPTOR/PTK-STAT proteins present in a cell.

47. The method of claim 46 wherein the treatment consists of administering an agent that increases the amount of phosphorylated RECEPTOR/PTK-STAT proteins present in a cell.

48. The method of claim 46 wherein the treatment consists of a gene-therapeutic method that increases the amount of phosphorylated RECEPTOR/PTK-STAT proteins present in a cell.

49. The method of claim 45 wherein the mammalian disease or developmental defect is selected from the group consisting of cancer and tumor cell metastasis.

50. A method of treating mammalian diseases or developmental defects caused by cell growth retardation wherein the method comprises promoting cell growth by altering the RECEPTOR/PTK-STAT pathway.

51. The method of claim 50 wherein the RECEPTOR/PTK-STAT pathway is altered by decreasing the amount of phosphorylated RECEPTOR/PTK-STAT proteins present in a cell.

52. The method of claim 51 wherein the treatment consists of administering an agent that decreases the amount of phosphorylated RECEPTOR/PTK-STAT proteins present in a cell.

53. The method of claim 51 wherein the treatment consists of a gene-therapeutic method that decreases the amount of phosphorylated RECEPTOR/PTK-STAT proteins present in a cell.

54. A method of treating mammalian diseases or developmental defects caused by abnormal cell detachment wherein the method comprises promoting cell attachment by altering the RECEPTOR/PTK-STAT pathway.

55. The method of claim 54 wherein the RECEPTOR/PTK-STAT pathway is altered by decreasing the amount of phosphorylated RECEPTOR/PTK-STAT proteins present in a cell.

56. The method of claim 55 wherein the treatment consists of administering an agent that decreases the amount of phosphorylated RECEPTOR/PTK-STAT proteins present in a cell.

57. The method of claim 55 wherein the treatment consists of a gene-therapeutic method that decreases the amount of phosphorylated RECEPTOR/PTK-STAT proteins present in a cell.

58. A method of treating mammalian diseases or developmental defects caused by abnormal cell detachment wherein the method comprises inhibiting cell attachment by altering the RECEPTOR/PTK-STAT pathway.

59. The method of claim 58 wherein the RECEPTOR/PTK-STAT pathway is altered by increasing the amount of phosphorylated RECEPTOR/PTK-STAT proteins present in a cell.

60. The method of claim 59 wherein the treatment consists of administering an agent that increases the amount of phosphorylated RECEPTOR/PTK-STAT proteins present in a cell.

61. The method of claim 59 wherein the treatment consists of a gene-therapeutic method that increases the amount of phosphorylated RECEPTOR/PTK-STAT proteins present in a cell.

62. A method for identifying diagnostic agents for measuring RECEPTOR/PTK-STAT activities in order to determine physiological and pathological conditions, wherein the method comprises the steps of:
a) measuring the activity of a RECEPTOR/PTK-STAT protein,
b) determining whether the activity of the RECEPTOR/PTK-STAT protein is associated with a specific phenotype or a specific disease, and
c). examining cellular localization of STAT protein to determine activation of STATs.

63. The method of claim 62 wherein the method is selected from the group consisting of in vivo assays and in vitro assays.

64. The method of claim 62 wherein the method is selected from the group consisting of measuring the activities of the RECEPTOR proteins, measuring the activities of the PTK proteins, and measuring the activities of the STAT proteins.

65. A clone that produces an exogenous level of STAT protein in an amount significantly greater than the parental cell line from which the clone was developed.

66. The clone of claim 65 wherein the STAT protein is selected from the group consisting of STAT1, STAT3, STAT4, STAT5A/B and STAT6.

67. The clone of claim 65 wherein the clone is selected from the group consisting of Ba/F3+STAT1, Ba/F3+STAT3 wt3, Ba/F3+STAT3 wt10, and U3A-STAT1.

68. The clone of claim 65 wherein the clone exhibits significantly faster cell death following serum withdrawal than the cell death of the parental cell line under the same conditions.

69. A method for identifying agents that block the phosphorylation of RECEPTOR/PTK-STAT comprising the steps of:
a) growing the clone of claim 65 in a serum-based growth media,
b) removing the serum from the growing media and concurrently adding the agent of interest,
c) determining whether said agent blocks the phosphorylation of RECEPTOR/PTK-STAT by observing clone cell viability over time.

70. A method of diagnosing abnormal STAT activation related to mammalian diseases comprising the steps of:
a) isolating and growing test cells from an individual of interest;
b) conducting nuclear staining of the test cells using anti-STAT antibodies;
c) examining the stained nuclei of the test cells to determine whether or not STAT has been translocated into the nuclei of the test cells; and,
d) comparing the extent of STAT translocation into the nuclei of the test cells to that of normal control cells stained in the same manner.

71. The method of claim 70 wherein the anti-STAT antibody is anti-STAT1.

72. The method of claim 70 wherein the mammalian disease is selected from the group consisting of Thanatophoric Dysplasia Type II, FGF-receptor associated diseases, cancer, metastasis of cancer cells, autoimmune disorders, diabetes, degenerative diseases, aging, and inflammation.

73. A method of determining the amount of phosphorylated STAT proteins wherein the method comprises using anti-phospho-tyrosine STAT.

74. The method of claim 73 wherein the anti-phospho-tyrosine STAT is anti-phospho-tyrosine STAT1.

75. The method of claim 73 wherein the method further comprising using Western blot analysis.

76. The method of claim 1 wherein the RECEPTOR/PTK-STAT pathway is altered by altering the interaction of STAT with a STAT DNA binding element.

77. The method of claim 1 wherein the RECEPTOR/PTK-STAT pathway is altered by altering the interaction among the RECEPTORs, the PTKs, and the STATs in the pathway.

78. The method of claim 1 wherein the RECEPTOR/PTK-STAT pathway is altered by altering the expression and activation of RECEPTOR, PTK, and STAT, either individually or in combination.

79. The method of claim 1 wherein the RECEPTOR/PTK-STAT pathway is altered by changing the RECEPTOR in the pathway.
